(19) United States
(12) Patent Application Publication
(10) Pub. No.: US 2016/0213039 A1
(43) Pub. Date: Jul. 28, 2016

(54) STEVIOL GLYCOSIDES

(71) Applicant: DSM IP ASSETS B.V., Heerlen (NL)

(72) Inventors: Manoj KUMAR, Echt (NL); Marieke NIJMEIJER, Echt (NL)

(73) Assignee: DSM IP ASSETS B.V., Heerlen (NL)

(21) Appl. No.: 14/908,146

(22) PCT Filed: Jul. 31, 2014
(86) PCT No.: PCT/EP2014/066555

§ 371 Date: Jan. 28, 2016

Related U.S. Application Data
(63) Continuation of application No. 13/956,226, filed on Jul. 31, 2013, now abandoned.

(51) Int. Cl. A23L 1/236; A23L 2/02; A21D 13/06; A21D 2/36; A23G 1/40; A23G 9/34; A61K 8/24; A61K 8/73; A61K 8/34; A61K 8/86; A61K 8/44; A61K 8/60; A23L 2/60; A61Q 11/00

(52) U.S. Cl. CPC A23L 1/2363; A23L 2/60; A23L 2/02; A21D 13/062; A21D 2/36; A23G 1/40; A23G 9/34; A23L 1/238; A61Q 11/00; A61K 8/24; A61K 8/73; A61K 8/731; A61K 8/345; A61K 8/738; A61K 8/86; A61K 8/442; A61K 8/602; A23V 2002/00; A61K 2800/59

(57) ABSTRACT

The present invention relates to a product which is a foodstuff, beverage, pharmaceutical composition, tobacco, nutraceutical, oral hygiene composition or cosmetic comprising a sweetener composition, wherein the sweetener composition comprises one or more fermentatively-produced steviol glycosides.

### FIELD OF THE INVENTION

The present invention relates to products comprising a sweetener composition and to a method for the preparation of such products. The invention further relates to the use of sweetener compositions in the preparation of the products and to a steviol-glycoside containing composition.

### BACKGROUND TO THE INVENTION

The use of artificial sweeteners such as dulcin, sodium cyclamate, aspartame, acylsulfame and saccharin has been seeing lower consumer demand for some time. However, non-caloric high potency sweeteners with natural origin such as steviol glycosides have been receiving increasing demand. In addition, the sweet steviol glycosides have functional and sensory properties superior to those of many high potency sweeteners.

Steviol glycosides (Masaya Ohta et al, J. Appl. Glycosci, v 57, (2010), pages 199-209) are a group of different sweet diterpene glycosides, which have a single base—steviol diterpene and differ by the presence of carbohydrate residues at positions C13 and C19.

The physical and sensory properties of Rebaudioside A and a number of other steviol glycosides have been studied (Caroline Hellfritsch et. al. J. Agric Food Chem, v60, (2012) pages 6782-6793). They have been tested for stability in carbonated beverages and found to be both heat and pH stable (Chang and Cook, 1983). The sweetness potency of Reb-A is between 200-400 times higher than sucrose,

However, apart from its high level of sweetness, they have also intrinsic properties of post-bitter taste and metallic and liquorish aftertaste. Some undesirable taste characteristics of steviol glycosides as a result of contamination of other substances such as polyphenolics and other flavor compound present in stevia plant extracts. There are thus limitations associated with the use of plant-derived steviol glycoside extracts.

### SUMMARY OF THE INVENTION

One of the main ways to improve the taste quality is the fermentative production of steviol glycosides. Another way to produce highly purified individual glycosides with desired clean taste characteristics and minimal content of accompanying compounds is to tailor make specific steviol glycosides via designed biosynthesis in a fermentation production host and fermentation thereof.

Accordingly, the invention is related to processes for microbial production of steviol glycosides by fermentation and to fermentatively-produced steviol glycosides, such as rebaudioside A (rebA), of certain product specification and use thereof. That is to say, the present invention relates to a microbial fermentation process for producing a highly purified rebA and to its use thereof in various food products and beverages.

A process for the fermentative production and recovery of diterpene glycosides, from microbial fermentation broth leading to a product specification is disclosed. In particular, a method for the recovery of rebA from microbial fermentation broth is described. Individual sweet glycosides can be obtained from a microbial fermentation process.

A mixture of sweet steviol glycosides can also be obtained from a designed microbial fermentation process and may be further processed to remove spent fermentation medium components by down-stream purification processes.

In contrast to plant-extracted steviol glycosides, fermentatively-produced rebA may readily be produced in a highly purified grade and in a form which has less residual bitterness and aftertaste.

Accordingly, the invention relates to a process of fermentative production of high purity rebA. The process is useful for producing high purity rebA with a product specification of at least about 95% rebA (dry weight basis).

This product specification of fermentatively-produced rebA is useful as a non-caloric sweetener in various food and beverage compositions as well as being useful in combination with other caloric and non-caloric sweeteners.

This fermentatively-produced reb-A is useful as a non-caloric sweetener in edible and chewable compositions such as any beverages, confectioneries, bakeries, cookies, chewing gums, and alike.

An object of the present invention is thus to provide a commercially valuable fermentative production process for producing a highly purified sweetener with known product specifications from a microbial production system and its use in various food products and beverages, overcoming the disadvantages of known plant extracted steviol glycosides.

In particular, fermentatively-produced rebA may be used to enhance citrus or sour attributes, total aroma impact, sweet aromatic complex, ethyl maltol (strawberry flavor) or brown fruit (flavor/aroma).

Herein is described a fermentation process where steviol glycosides are produced via microbial fermentation. Also described is a down-stream purification and recovery process for recovery of fermentatively-produced steviol glycosides along with a highly-purified rebA of a given product specification.

The fermentatively produced steviol glycoside (of specific product specification) may be applied in various foods and beverages as a sweetener. That is to say, the present invention is based on fermentatively-produced steviol glycosides suitable for various food and beverage applications.

According to the invention, there is thus provided a fermentatively derived product which is a foodstuff, beverage, pharmaceutical composition, tobacco, nutraceutical, oral hygiene composition or cosmetic comprising a sweetener composition, wherein the sweetener composition comprises one or more fermentatively-produced steviol glycosides.

The invention also provides a method for the preparation of a product which is a foodstuff, beverage, pharmaceutical composition, tobacco, nutraceutical, oral hygiene composition or cosmetic comprising a sweetener composition, which method comprises preparing a said product and incorporating a sweetener composition comprising one or more fermentatively-produced steviol glycosides. Such a method may also comprise fermenting a microorganism as herein described and recovering rebaudioside A from the microorganism and/or extracellular medium.

Further provided by the invention is use of a sweetener composition comprising one or more fermentatively-produced steviol glycosides in the preparation of a foodstuff, beverage, pharmaceutical composition, tobacco, nutraceutical, oral hygiene composition or cosmetic comprising a sweetener composition.

Also provided by the invention is a composition which comprises, on a dry solids basis, at least about 95% fermentatively-produced Rebaudioside A.

### BRIEF DESCRIPTION OF THE DRAWINGS

FIG. 1 sets out a schematic representation of the plasmid pUG7-EcoRV.

FIG. 2 sets out a schematic representation of the method by which the ERG20, tHMG1 and BTS1 over-expression cassettes are designed (A) and integrated (B) into the yeast genome. (C) shows the final situation after removal of the KANMX marker by the Cre recombinase.

FIG. 3 sets out a schematic representation of the ERG9 knock down construct. This consists of a 500 bp long 3′ part of ERG9, 98 bp of the TRP1 promoter, the TRP1 open reading frame and terminator, followed by a 400 bp long downstream sequence of ERG9. Due to introduction of a XbaI site at the end of the ERG9 open reading frame the last amino acid changes into Ser and the stop codon into Arg. A new stop codon is located in the TPR1 promoter, resulting in an extension of 18 amino acids.

FIG. 4 sets out a schematic representation of how UGT2 is integrated into the genome. A. different fragments used in transformation; B. situation after integration; C. situation after expression of Cre recombinase.

FIG. 5 sets out a schematic representation of how the pathway from GGPP to RebA is integrated into the genome. A. different fragments used in transformation; B. situation after integration.

FIG. 6 sets out a schematic diagram of the potential pathways leading to biosynthesis of steviol glycosides.

FIG. 7 sets out a process for recovery of steviol glycosides from a fermentation broth.

FIG. 8 shows sweetness intensity score per application (acidified water, near water, juice and cola), fermentative Reb A versus Plant based Reb A.

FIG. 9 shows the intensity scores for fermentative Reb A versus Plant based Reb A in acidified water

FIG. 10 shows the intensity scores fermentative Reb A versus Plant based Reb A in near water.

FIG. 11 shows the intensity scores fermentative Reb A versus Plant based Reb A in juice.

### DESCRIPTION OF THE SEQUENCE LISTING

A description of the sequences is set out in Table 1. Sequences described herein may be defined with reference to the sequence listing or with reference to the database accession numbers also set out in Table 1.

### DETAILED DESCRIPTION OF THE INVENTION

Advantages of the present invention will become more apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

Throughout the present specification and the accompanying claims, the words “comprise”, “include” and “having” and variations such as “comprises”, “comprising”, “includes” and “including” are to be interpreted inclusively. That is, these words are intended to convey the possible inclusion of other elements or integers not specifically recited, where the context allows.

The invention relates to products comprising sweetener compositions. The sweetener compositions comprise one or more steviol glycosides, one or more of which is prepared fermentatively.

The invention thus provides a solution comprising one or more steviol glycosides. Such a solution may comprise rebaudioside A.

Such a solution may comprise, on a dry solids basis, at least about 60%, at least about 70%, at least about 80%, at least about 90%, at least about 95%, at least about 99% weight of Rebaudioside A.

Accordingly, the invention provides a composition which may comprise, on a dry solids basis, at least about 60%, at least about 70%, at least about 80%, at least about 90%, at least about 95%, at least about 99% weight of fermentatively-produced Rebaudioside A.

Such a composition may be a granulate or powder. Such a solid composition may comprise at least about 60%, at least about 70%, at least about 80%, at least about 90%, at least about 95%, at least about 99% by weight of fermentatively-produced Rebaudioside A.

Such solutions and compositions may be prepared by fermentation of a recombinant microorganism that is capable of producing a steviol glycoside. Suitable recombinant microorganisms are described herein below. Such a recombinant microorganism may comprise one or more nucleotide sequence(s) encoding:
a polypeptide having ent-copalyl pyrophosphate synthase activity;
a polypeptide having ent-Kaurene synthase activity;
a polypeptide having ent-Kaurene oxidase activity;
a polypeptide having kaurenoic acid 13-hydroxylase activity; and
one or more polypeptides having UDP-glucosyltransferase (UGT) activity,

whereby expression of the nucleotide sequence(s) confer(s) on the microorganism the ability to produce at least one steviol glycoside.

For the purposes of this invention, a polypeptide having ent-copalyl pyrophosphate synthase (EC 5.5.1.13) is capable of catalyzing the chemical reaction:

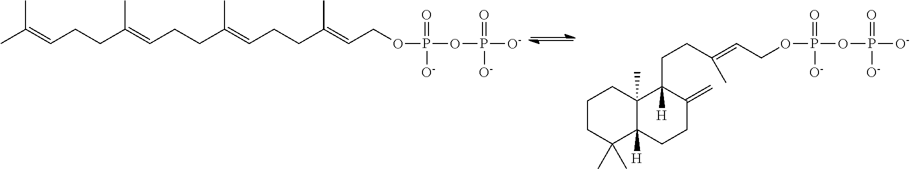

This enzyme has one substrate, geranylgeranyl pyrophosphate, and one product, ent-copalyl pyrophosphate. This enzyme participates in gibberellin biosynthesis. This enzyme belongs to the family of isomerase, specifically the class of intramolecular lyases. The systematic name of this enzyme class is ent-copalyl-diphosphate lyase (decyclizing). Other names in common use include having ent-copalyl pyrophosphate synthase, ent-kaurene synthase A, and ent-kaurene synthetase A.

For the purposes of this invention, a polypeptide having ent-kaurene synthase activity (EC 4.2.3.19) is a polypeptide that is capable of catalyzing the chemical reaction:

ent-copalyl diphosphate [symbol] ent-kaurene+diphosphate

Hence, this enzyme has one substrate, ent-copalyl diphosphate, and two products, ent-kaurene and diphosphate.

This enzyme belongs to the family of lyases, specifically those carbon-oxygen lyases acting on phosphates. The systematic name of this enzyme class is ent-copalyl-diphosphate diphosphate-lyase (cyclizing, ent-kaurene-forming). Other names in common use include ent-kaurene synthase B, ent-kaurene synthetase B, ent-copalyl-diphosphate diphosphate-lyase, and (cyclizing). This enzyme participates in diterpenoid biosynthesis.

ent-copalyl diphosphate synthases may also have a distinct ent-kaurene synthase activity associated with the same protein molecule. The reaction catalyzed by ent-kaurene synthase is the next step in the biosynthetic pathway to gibberellins. The two types of enzymic activity are distinct, and site-directed mutagenesis to suppress the ent-kaurene synthase activity of the protein leads to build up of ent-copalyl pyrophosphate.

Accordingly, a single nucleotide sequence may encode a polypeptide having ent-copalyl pyrophosphate synthase activity and ent-kaurene synthase activity. Alternatively, the two activities may be encoded two distinct, separate nucleotide sequences.

For the purposes of this invention, a polypeptide having ent-kaurene oxidase activity (EC 1.14.13.78) is a polypeptide which is capable of catalysing three successive oxidations of the 4-methyl group of ent-kaurene to give kaurenoic acid. Such activity typically requires the presence of a cytochrome P450.

For the purposes of the invention, a polypeptide having kaurenoic acid 13-hydroxylase activity (EC 1.14.13) is one which is capable of catalyzing the formation of steviol (ent-kaur-16-en-13-ol-19-oic acid) using NADPH and O₂. Such activity may also be referred to as ent-ka 13-hydroxylase activity.

A recombinant microorganism which may be fermented to produce a fermentation broth for use in the process of the invention comprises one or more nucleotide sequences encoding a polypeptide having UDP-glucosyltransferase (UGT) activity, whereby expression of the nucleotide sequence(s) confer(s) on the microorganism the ability to produce at least one of steviolmonoside, steviolbioside, stevioside or rebaudioside A, rebaudioside B, rebaudioside C, rebaudioside D, rebaudioside E, rebaudioside F, rubusoside, dulcoside A or rebaudioside M.

For the purposes of this invention, a polypeptide having UGT activity is one which has glycosyltransferase activity (EC 2.4), i.e. that can act as a catalyst for the transfer of a monosaccharide unit from an activated nucleotide sugar (also known as the “glycosyl donor”) to a glycosyl acceptor molecule, usually an alcohol. The glycosyl donor for a UGT is typically the nucleotide sugar uridine diphosphate glucose (uracil-diphosphate glucose, UDP-glucose).

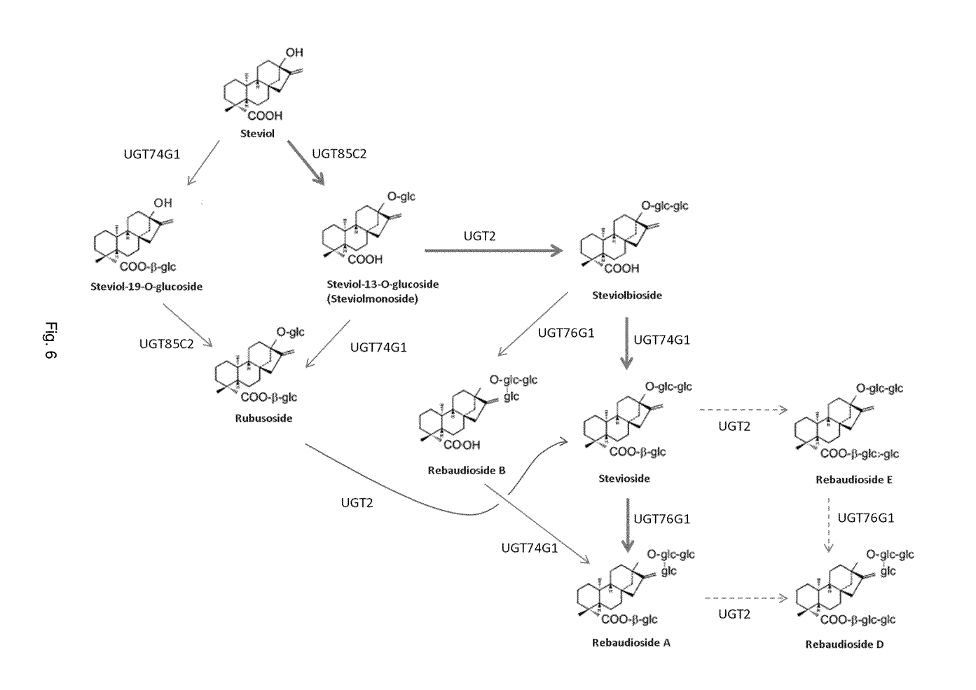

The UGTs used may be selected so as to produce a desired diterpene glycoside, such as a steviol glycoside. Schematic diagrams of steviol glycoside formation are set out in Humphrey et al., Plant Molecular Biology (2006) 61: 47-62 and Mohamed et al., J. Plant Physiology 168 (2011) 1136-1141. In addition, FIG. 6 sets out a schematic diagram of steviol glycoside formation.

The biosynthesis of rebaudioside A involves glucosylation of the aglycone steviol. Specifically, rebaudioside A can be formed by glucosylation of the 13-OH of steviol which forms the 13-O-steviolmonoside, glucosylation of the C-2′ of the 13-O-glucose of steviolmonoside which forms steviol-1,2-bioside, glucosylation of the C-19 carboxyl of steviol-1,2-bioside which forms stevioside, and glucosylation of the C-3′ of the C-13-O-glucose of stevioside. The order in which each glucosylation reaction occurs can vary—see FIG. 6. One UGT may be capable of catalyzing more than one conversion as set out in this scheme.

Conversion of steviol to rebaudioside A or rebaudioside D may be accomplished in a recombinant host by the expression of gene(s) encoding the following functional UGTs: UGT74G1, UGT85C2, UGT76G1 and UGT2. Thus, a recombinant microorganism expressing these four UGTs can make rebaudioside A if it produces steviol or when fed steviol in the medium. Typically, one or more of these genes are recombinant genes that have been transformed into a microorganism that does not naturally possess them. Examples of all of these enzmyes are set out in Table 1. A recombinant microorganism may comprise any combination of a UGT74G1, UGT85C2, UGT76G1 and UGT2. In Table 1 UGT64G1 sequences are indicated as UGT1 sequences, UGT74G1 sequences are indicated as UGT3 sequences and UGT76G1 sequences are indicated as UGT4 sequences. UGT2 sequences are indicated as UGT2 sequences in Table 1.

A recombinant microorganism which comprises a nucleotide sequence encoding a polypeptide having UGT activity may comprise a nucleotide sequence encoding a polypeptide capable of catalyzing the addition of a C-13-glucose to steviol. That is to say, a recombinant microorganism may comprise a UGT which is capable of catalyzing a reaction in which steviol is converted to steviolmonoside. Accordingly, expression of such a nucleotide sequence may confer on the microorganism the ability to produce at least steviolmonoside.

Such a microorganism may comprise a nucleotide sequence encoding a polypeptide having the activity shown by UDP-glycosyltransferase (UGT) UGT85C2, whereby the nucleotide sequence upon transformation of the microorganism confers on the cell the ability to convert steviol to steviolmonoside.

UGT85C2 activity is transfer of a glucose unit to the 13-OH of steviol. Thus, a suitable UGT85C2 may function as a uridine 5′-diphospho glucosyl: steviol 13-OH transferase, and a uridine 5′-diphospho glucosyl: steviol-19-0-glucoside 13-OH transferase. A functional UGT85C2 polypeptides may also catalyze glucosyl transferase reactions that utilize steviol glycoside substrates other than steviol and steviol-19-O-glucoside. Such sequences are indicated as UGT1 sequences in Table 1.

A recombinant microorganism which comprises a nucleotide sequence encoding a polypeptide having UGT activity may comprise a nucleotide sequence encoding a polypeptide capable of catalyzing the addition of a C-13-glucose to steviol or steviolmonoside. That is to say, a recombinant microorganism may comprise a UGT which is capable of catalyzing a reaction in which steviolmonoside is converted to steviolbioside. Accordingly, such a microorganism may be capable of converting steviolmonoside to steviolbioside. Expression of such a nucleotide sequence may confer on the microorganism the ability to produce at least steviolbioside.

A suitable recombinant microorganism may also comprise a nucleotide sequence encoding a polypeptide having the activity shown by UDP-glycosyltransferase (UGT) UGT74G1, whereby the nucleotide sequence upon transformation of the microorganism confers on the cell the ability to convert steviolmonoside to steviolbioside.

A suitable recombinant microorganism may also comprise a nucleotide sequence encoding a polypeptide having the activity shown by UDP-glycosyltransferase (UGT) UGT2, whereby the nucleotide sequence upon transformation of the microorganism confers on the cell the ability to convert steviolmonoside to steviolbioside.

A suitable UGT2 polypeptide functions as a uridine 5′-diphospho glucosyl: steviol-13-O-glucoside transferase (also referred to as a steviol-13-monoglucoside 1,2-glucosylase), transferring a glucose moiety to the C-2′ of the 13-0-glucose of the acceptor molecule, steviol-13-O-glucoside. Typically, a suitable UGT2 polypeptide also functions as a uridine 5′-diphospho glucosyl: rubusoside transferase transferring a glucose moiety to the C-2′ of the 13-O-glucose of the acceptor molecule, rubusoside.

Functional UGT2 polypeptides may also catalyze reactions that utilize steviol glycoside substrates other than steviol-13-O-glucoside and rubusoside, e.g., functional UGT2 polypeptides may utilize stevioside as a substrate, transferring a glucose moiety to the C-2′ of the 19-O-glucose residue to produce Rebaudioside E. A functional UGT2 polypeptides may also utilize Rebaudioside A as a substrate, transferring a glucose moiety to the C-2′ of the 19-O-glucose residue to produce Rebaudioside D. However, a functional UGT2 polypeptide typically does not transfer a glucose moiety to steviol compounds having a 1,3-bound glucose at the C-13 position, i.e., transfer of a glucose moiety to steviol 1,3-bioside and 1,3-stevioside does not occur.

Functional UGT2 polypeptides may also transfer sugar moieties from donors other than uridine diphosphate glucose. For example, a functional UGT2 polypeptide may act as a uridine 5′-diphospho D-xylosyl: steviol-13-O-glucoside transferase, transferring a xylose moiety to the C-2′ of the 13-O-glucose of the acceptor molecule, steviol-13-O-glucoside. As another example, a functional UGT2 polypeptide can act as a uridine 5′-diphospho L-rhamnosyl: steviol-13-O-glucoside transferase, transferring a rhamnose moiety to the C-2′ of the 13-O-glucose of the acceptor molecule, steviol-13-O-glucoside. Such sequences are indicated as UGT2 sequences in Table 1.

A recombinant microorganism which may be fermented to produce a fermentation broth for use in a process of the invention which comprises a nucleotide sequence encoding a polypeptide having UGT activity may comprise a nucleotide sequence encoding a polypeptide capable of catalyzing the addition of a C-19-glucose to steviolbioside. That is to say, a suitable recombinant microorganism may comprise a UGT which is capable of catalyzing a reaction in which steviolbioside is converted to stevioside. Accordingly, such a microorganism may be capable of converting steviolbioside to stevioside. Expression of such a nucleotide sequence may confer on the microorganism the ability to produce at least stevioside.

A suitable recombinant microorganism may also comprise a nucleotide sequence encoding a polypeptide having the activity shown by UDP-glycosyltransferase (UGT) UGT74G1, whereby the nucleotide sequence upon transformation of the microorganism confers on the cell the ability to convert steviolbioside to stevioside.

Suitable UGT74G1 polypeptides may be capable of transferring a glucose unit to the 13-OH or the 19-000H, respectively, of steviol. A suitable UGT74G1 polypeptide may function as a uridine 5′-diphospho glucosyl: steviol 19-000H transferase and a uridine 5′-diphospho glucosyl: steviol-13-O-glucoside 19-000H transferase. Functional UGT74G1 polypeptides also may catalyze glycosyl transferase reactions that utilize steviol glycoside substrates other than steviol and steviol-13-O-glucoside, or that transfer sugar moieties from donors other than uridine diphosphate glucose. Such sequences are indicated as UGT1 sequences in Table 3.

A recombinant microorganism which comprises a nucleotide sequence encoding a polypeptide having UGT activity may comprise a nucleotide sequence encoding a polypeptide capable of catalyzing glucosylation of the C-3′ of the glucose at the C-13 position of stevioside. That is to say, a recombinant microorganism may comprise a UGT which is capable of catalyzing a reaction in which stevioside to rebaudioside A. Accordingly, such a microorganism may be capable of converting stevioside to rebaudioside A. Expression of such a nucleotide sequence may confer on the microorganism the ability to produce at least rebaudioside A.

A suitable recombinant microorganism may also comprise a nucleotide sequence encoding a polypeptide having the activity shown by UDP-glycosyltransferase (UGT) UGT76G1, whereby the nucleotide sequence upon transformation of the microorganism confers on the cell the ability to convert stevioside to rebaudioside A.

A suitable UGT76G1 adds a glucose moiety to the C-3′ of the C-13-O-glucose of the acceptor molecule, a steviol 1,2 glycoside. Thus, UGT76G1 functions, for example, as a uridine 5′-diphospho glucosyl: steviol 13-O-1,2 glucoside C-3 ‘ glucosyl transferase and a uridine 5’-diphospho glucosyl: steviol-19-O-glucose, 13-O-1,2 bioside C-3′ glucosyl transferase. Functional UGT76G1 polypeptides may also catalyze glucosyl transferase reactions that utilize steviol glycoside substrates that contain sugars other than glucose, e.g., steviol rhamnosides and steviol xylosides. Such sequences are indicated as UGT4 sequences in Table 1.

A recombinant microorganism may comprise nucleotide sequences encoding polypeptides having one or more of the four UGT activities described above. Preferably, a recombinant microorganism may comprise nucleotide sequences encoding polypeptides having all four of the UGT activities described above. A given nucleic acid may encode a polypeptide having one or more of the above activities. For example, a nucleic acid encode for a polypeptide which has two, three or four of the activities set out above. Preferably, a recombinant microorganism comprises UGT1, UGT2 and UGT3 activity. More preferably, such a recombinant microorganism will also comprise UGT4 activity.

A recombinant microorganism which comprises a nucleotide sequence encoding a polypeptide having UGT activity may comprise a nucleotide sequence encoding a polypeptide capable of catalyzing the glucosylation of stevioside or rebaudioside A. That is to say, a recombinant microorganism may comprise a UGT which is capable of catalyzing a reaction in which stevioside or rebaudioside A is converted to rebaudioside D. Accordingly, such a microorganism may be capable of converting stevioside or rebaudioside A to rebaudioside D. Expression of such a nucleotide sequence may confer on the microorganism the ability to produce at least rebaudioside D. We have shown that a microorganism expression a combination of UGT85C2, UGT2, UGT74G1 and UGT76G1 polypeptides may be capable of rebaudioside D production.

A microorganism which comprises a nucleotide sequence encoding a polypeptide having UGT activity may comprise a nucleotide sequence encoding a polypeptide capable of catalyzing the glucosylation of stevioside. That is to say, a microorganism may comprise a UGT which is capable of catalyzing a reaction in which stevioside is converted to rebaudioside E. Accordingly, such a microorganism may be capable of converting stevioside to rebaudioside E. Expression of such a nucleotide sequence may confer on the microorganism the ability to produce at least rebaudioside E.

A microorganism which comprises a nucleotide sequence encoding a polypeptide having UGT activity may comprise a nucleotide sequence encoding a polypeptide capable of catalyzing the glucosylation of rebaudioside E. That is to say, a microorganism may comprise a UGT which is capable of catalyzing a reaction in which rebaudioside E is converted to rebaudioside D. Accordingly, such a microorganism may be capable of converting stevioside or rebaudioside A to rebaudioside D. Expression of such a nucleotide sequence may confer on the microorganism the ability to produce at least rebaudioside D.

A recombinant microorganism may be capable of expressing a nucleotide sequence encoding a polypeptide having NADPH-cytochrome p450 reductase activity. That is to say, a recombinant microorganism may comprise sequence encoding a polypeptide having NADPH-cytochrome p450 reductase activity.

A polypeptide having NADPH-Cytochrome P450 reductase activity (EC 1.6.2.4; also known as NADPH:ferrihemoprotein oxidoreductase, NADPH:hemoprotein oxidoreductase, NADPH:P450 oxidoreductase, P450 reductase, POR, CPR, CYPOR) is typically one which is a membrane-bound enzyme allowing electron transfer to cytochrome P450 in the microsome of the eukaryotic cell from a FAD- and FMN-containing enzyme NADPH:cytochrome P450 reductase (POR; EC 1.6.2.4).

Preferably, a recombinant microorganism, capable of being fermented to prepare a fermentation broth suitable for use in the process of the invention, is capable of expressing one or more of:
a. a nucleotide sequence encoding a polypeptide having NADPH-cytochrome p450 reductase activity, wherein said nucleotide sequence comprises:
   (a) a nucleotide sequence encoding a polypeptide having NADPH-cytochrome p450 reductase activity, said polypeptide comprising an amino acid sequence that has at least about 20%, preferably at least 25, 30, 40, 50, 55, 60, 65, 70, 75, 80, 85, 90, 95, 96, 97, 98, or 99%, sequence identity with the amino acid sequence of SEQ ID NOs: 54, 56, 58 or 78;
   (b) a nucleotide sequence that has at least about 15%, preferably at least 20, 25, 30, 40, 50, 55, 60, 65, 70, 75, 80, 85, 90, 95, 96, 97, 98, or 99%, sequence identity with the nucleotide sequence of SEQ ID NOs: 53, 55, 57 or 77;
   (c) a nucleotide sequence the complementary strand of which hybridizes to a nucleic acid molecule of sequence of (i) or (ii); or
   (d) a nucleotide sequence which differs from the sequence of a nucleic acid molecule of (i), (ii) or (iii) due to the degeneracy of the genetic code,

Preferably, a recombinant microorganism is one which is capable of expressing one or more of:
a. a nucleotide sequence encoding a polypeptide having ent-copalyl pyrophosphate synthase activity, wherein said nucleotide sequence comprises:
   1. a nucleotide sequence encoding a polypeptide having ent-copalyl pyrophosphate synthase activity, said polypeptide comprising an amino acid sequence that has at least about 20%, preferably at least 25, 30, 40, 50, 55, 60, 65, 70, 75, 80, 85, 90, 95, 96, 97, 98, or 99%, sequence identity with the amino acid sequence of SEQ ID NOs: 2, 4, 6, 8, 18, 20, 60 or 62;
   2. a nucleotide sequence that has at least about 15%, preferably at least 20, 25, 30, 40, 50, 55, 60, 65, 70, 75, 80, 85, 90, 95, 96, 97, 98, or 99%, sequence identity with the nucleotide sequence of SEQ ID NOs: 1, 3, 5, 7, 17, 19, 59 or 61, 141, 142, 151, 152, 153, 154, 159, 160, 182 or 184;
   3. a nucleotide sequence the complementary strand of which hybridizes to a nucleic acid molecule of sequence of (i) or (ii); or
   4. a nucleotide sequence which differs from the sequence of a nucleic acid molecule of (i), (ii) or (iii) due to the degeneracy of the genetic code,
b. a nucleotide sequence encoding a polypeptide having ent-Kaurene synthase activity, wherein said nucleotide sequence comprises:
   1. a nucleotide sequence encoding a polypeptide having ent-Kaurene synthase activity, said polypeptide comprising an amino acid sequence that has at least about 20%, preferably at least 25, 30, 40, 50, 55, 60, 65, 70, 75, 80, 85, 90, 95, 96, 97, 98, or 99%, sequence identity with the amino acid sequence of SEQ ID NOs: 10, 12, 14, 16, 18, 20, 64 or 66;
   2. a nucleotide sequence that has at least about 15%, preferably at least 20, 25, 30, 40, 50, 55, 60, 65, 70, 75, 80, 85, 90, 95, 96, 97, 98, or 99%, sequence identity with the nucleotide sequence of SEQ ID NOs: 9, 11, 13, 15, 17, 19, 63, 65, 143, 144, 155, 156, 157, 158, 159, 160, 183 or 184;
   3. a nucleotide sequence the complementary strand of which hybridizes to a nucleic acid molecule of sequence of (i) or (ii); or
   4. a nucleotide sequence which differs from the sequence of a nucleic acid molecule of (i), (ii) or (iii) due to the degeneracy of the genetic code,
c. a nucleotide sequence encoding a polypeptide having ent-Kaurene oxidase activity, wherein said nucleotide sequence comprises:
   1. a nucleotide sequence encoding a polypeptide having ent-Kaurene oxidase activity, said polypeptide comprising an amino acid sequence that has at least about 20%, preferably at least 25, 30, 40, 50, 55, 60, 65, 70, 75, 80, 85, 90, 95, 96, 97, 98, or 99%, sequence identity with the amino acid sequence of SEQ ID NOs: 22, 24, 26, 68 or 86;
   2. a nucleotide sequence that has at least about 15%, preferably at least 20, 25, 30, 40, 50, 55, 60, 65, 70, 75, 80, 85, 90, 95, 96, 97, 98, or 99%, sequence identity with the nucleotide sequence of SEQ ID NOs: 21, 23, 25, 67, 85, 145, 161, 162, 163, 180 or 186;
   3. a nucleotide sequence the complementary strand of which hybridizes to a nucleic acid molecule of sequence of (i) or (ii); or
   4. a nucleotide sequence which differs from the sequence of a nucleic acid molecule of (i), (ii) or (iii) due to the degeneracy of the genetic code; or
d. a nucleotide sequence encoding a polypeptide having kaurenoic acid 13-hydroxylase activity, wherein said nucleotide sequence comprises:
   1 a nucleotide sequence encoding a polypeptide having kaurenoic acid 13-hydroxylase activity, said polypeptide comprising an amino acid sequence that has at least about 20%, preferably at least 25, 30, 40, 50, 55, 60, 65, 70, 75, 80, 85, 90, 95, 96, 97, 98, or 99%, sequence identity with the amino acid sequence of SEQ ID NOs: 28, 30, 32, 34, 70, 90, 92, 94, 96 or 98;
   2 a nucleotide sequence that has at least about 15%, preferably at least 20, 25, 30, 40, 50, 55, 60, 65, 70, 75, 80, 85, 90, 95, 96, 97, 98, or 99%, sequence identity with the nucleotide sequence of SEQ ID NOs: 27, 29, 31, 33, 69, 89, 91, 93, 95, 97, 146, 164, 165, 166, 167 or 185;
   3 a nucleotide sequence the complementary strand of which hybridizes to a nucleic acid molecule of sequence of (i) or (ii); or
   4 a nucleotide sequence which differs from the sequence of a nucleic acid molecule of (i), (ii) or (iii) due to the degeneracy of the genetic code.

In a recombinant microorganism which is capable of expressing a nucleotide sequence encoding a polypeptide capable of catalyzing the addition of a C-13-glucose to steviol, said nucleotide may comprise:
a nucleotide sequence encoding a polypeptide capable of catalyzing the addition of a C-13-glucose to steviol, said polypeptide comprising an amino acid sequence that has at least about 20%, preferably at least 25, 30, 40, 50, 55, 60, 65, 70, 75, 80, 85, 90, 95, 96, 97, 98, or 99%, sequence identity with the amino acid sequence of SEQ ID NOs: 36, 38 or 72;
a nucleotide sequence that has at least about 15%, preferably at least 20, 25, 30, 40, 50, 55, 60, 65, 70, 75, 80, 85, 90, 95, 96, 97, 98, or 99%, sequence identity with the nucleotide sequence of SEQ ID NOs: 35, 37, 71, 147, 168, 169 or 189;
a nucleotide sequence the complementary strand of which hybridizes to a nucleic acid molecule of sequence of (i) or (ii); or
a nucleotide sequence which differs from the sequence of a nucleic acid molecule of (i), (ii) or (iii) due to the degeneracy of the genetic code.

In a recombinant microorganism which is capable of expressing a nucleotide sequence encoding a polypeptide capable of catalyzing the addition of a glucose at the C-13 position of steviolmonoside (this typically indicates glucosylation of the C-2′ of the C-13-glucose/13-O-glucose of steviolmonoside), said nucleotide sequence may comprise:
1. a nucleotide sequence encoding a polypeptide capable of catalyzing the addition of a C-13-glucose to steviol or steviolmonoside, said polypeptide comprising an amino acid sequence that has at least about 20%, preferably at least 25, 30, 40, 50, 55, 60, 65, 70, 75, 80, 85, 90, 95, 96, 97, 98, or 99%, sequence identity with the amino acid sequence of SEQ ID NOs: 88, 100, 102, 104, 106, 108, 110 or 112;
2. a nucleotide sequence that has at least about 15%, preferably at least 20, 25, 30, 40, 50, 55, 60, 65, 70, 75, 80, 85, 90, 95, 96, 97, 98, or 99%, sequence identity with the nucleotide sequence of SEQ ID NOs: 87, 99, 101, 103, 105, 107, 109, 111, 181 or 192;
3. a nucleotide sequence the complementary strand of which hybridizes to a nucleic acid molecule of sequence of (i) or (ii); or
4. a nucleotide sequence which differs from the sequence of a nucleic acid molecule of (i), (ii) or (iii) due to the degeneracy of the genetic code.

In a recombinant microorganism which is capable of expressing a nucleotide sequence encoding a polypeptide capable of catalyzing the addition of a glucose at the C-19 position of steviolbioside, said nucleotide sequence may comprise:
1. a nucleotide sequence encoding a polypeptide capable of catalyzing the addition of a glucose at the C-19 position of steviolbioside, said polypeptide comprising an amino acid sequence that has at least about 20% sequence identity with the amino acid sequence of SEQ ID NOs: 40, 42, 44, 46, 48 or 74;
2. a nucleotide sequence that has at least about 15% sequence identity with the nucleotide sequence of SEQ ID NOs: 39, 41, 43, 45, 47, 73, 148, 170, 171, 172, 173, 174 or 190;
3. a nucleotide sequence the complementary strand of which hybridizes to a nucleic acid molecule of sequence of (i) or (ii); or
4. a nucleotide sequence which differs from the sequence of a nucleic acid molecule of (i), (ii) or (iii) due to the degeneracy of the genetic code.

In a recombinant microorganism which expresses a nucleotide sequence encoding a polypeptide capable of catalyzing glucosylation of the C-3′ of the glucose at the C-13 position of stevioside, said nucleotide sequence may comprise:
1. a nucleotide sequence encoding a polypeptide capable of catalyzing glucosylation of the C-3′ of the glucose at the C-13 position of stevioside, said polypeptide comprising an amino acid sequence that has at least about 20%, preferably at least 25, 30, 40, 50, 55, 60, 65, 70, 75, 80, 85, 90, 95, 96, 97, 98, or 99%, sequence identity with the amino acid sequence of SEQ ID NOs: 50, 52 or 76;
2. a nucleotide sequence that has at least about 15%, preferably at least 20, 25, 30, 40, 50, 55, 60, 65, 70, 75, 80, 85, 90, 95, 96, 97, 98, or 99%, sequence identity with the nucleotide sequence of SEQ ID NOs: 49, 51, 75, 149, 175, 176 or 191;
3. a nucleotide sequence the complementary strand of which hybridizes to a nucleic acid molecule of sequence of (i) or (ii); or
4. a nucleotide sequence which differs from the sequence of a nucleic acid molecule of (i), (ii) or (iii) due to the degeneracy of the genetic code.

In a recombinant microorganism which expresses a nucleotide sequence encoding a polypeptide capable of catalysing one or more of: the glucosylation of stevioside or rebaudioside A to rebaudioside D; the glucosylation of stevioside to rebaudioside E; or the glucosylation of rebaudioside E to rebaudioside D, said nucleotide sequence may comprise:
i. a nucleotide sequence encoding a polypeptide capable of catalysing one or more of: the glucosylation of stevioside or rebaudioside A to rebaudioside D; the glucosylation of stevioside to rebaudioside E; or the glucosylation of rebaudioside E to rebaudioside D, said polypeptide comprising an amino acid sequence that has at least about 20% sequence identity with the amino acid sequence of SEQ ID NOs: 88, 100, 102, 104, 106, 108, 110, 112;
ii. a nucleotide sequence that has at least about 15% sequence identity with the nucleotide sequence of SEQ ID NOs: 87, 99, 101, 103, 105, 107, 109, 111, 181 or 192;
iii. a nucleotide sequence the complementary strand of which hybridizes to a nucleic acid molecule of sequence of (i) or (ii); or
iv. a nucleotide sequence which differs from the sequence of a nucleic acid molecule of (i), (ii) or (iii) due to the degeneracy of the genetic code.

A suitable microorganism may be one in which the ability of the microorganism to produce geranylgeranyl pyrophosphate (GGPP) is upregulated. Upregulated in the context of this invention implies that the microorganism produces more GGPP than an equivalent non-transformed strain.

Accordingly, a suitable recombinant microorganism may comprise one or more nucleotide sequence(s) encoding hydroxymethylglutaryl-CoA reductase, farnesyl-pyrophosphate synthetase and geranylgeranyl diphosphate synthase, whereby the nucleotide sequence(s) upon transformation of the microorganism confer(s) on the microorganism the ability to produce elevated levels of GGPP.

Preferably, a suitable recombinant microorganism is one which is capable of expressing one or more of:
a. a nucleotide sequence encoding a polypeptide having hydroxymethylglutaryl-CoA reductase activity, wherein said nucleotide sequence comprises:
   1. a nucleotide sequence encoding a polypeptide having hydroxymethylglutaryl-CoA reductase activity, said polypeptide comprising an amino acid sequence that has at least about 20% sequence identity with the amino acid sequence of SEQ ID NO: 80;
   2. a nucleotide sequence that has at least about 15% sequence identity with the nucleotide sequence of SEQ ID NO: 79;
   3. a nucleotide sequence the complementary strand of which hybridizes to a nucleic acid molecule of sequence of (i) or (ii); or
   4. a nucleotide sequence which differs from the sequence of a nucleic acid molecule of (i), (ii) or (iii) due to the degeneracy of the genetic code,
b. a nucleotide sequence encoding a polypeptide having farnesyl-pyrophosphate synthetase activity, wherein said nucleotide sequence comprises:
   1. a nucleotide sequence encoding a polypeptide having farnesyl-pyrophosphate synthetase activity, said polypeptide comprising an amino acid sequence that has at least about 20% sequence identity with the amino acid sequence of SEQ ID NO: 82;
   2. a nucleotide sequence that has at least about 15% sequence identity with the nucleotide sequence of SEQ ID NOs: 81;
   3. a nucleotide sequence the complementary strand of which hybridizes to a nucleic acid molecule of sequence of (i) or (ii); or
   4. a nucleotide sequence which differs from the sequence of a nucleic acid molecule of (iii) due to the degeneracy of the genetic code; or
c. a nucleotide sequence encoding a polypeptide having geranylgeranyl diphosphate synthase activity, wherein said nucleotide sequence comprises:
   a nucleotide sequence encoding a polypeptide having geranylgeranyl diphosphate synthase activity, said polypeptide comprising an amino acid sequence that has at least about 20% sequence identity with the amino acid sequence of SEQ ID NO: 84;
   a nucleotide sequence that has at least about 15% sequence identity with the nucleotide sequence of SEQ ID NOs: 83;
   a nucleotide sequence the complementary strand of which hybridizes to a nucleic acid molecule of sequence of (i) or (ii); or
   a nucleotide sequence which differs from the sequence of a nucleic acid molecule of (i), (ii) or (iii) due to the degeneracy of the genetic code.

A microorganism or microbe, for the purposes of this invention, is typically an organism that is not visible to the human eye (i.e. microscopic). A microorganism may be from bacteria, fungi, archaea or protists. Typically a microorganism will be a single-celled or unicellular organism.

As used herein a recombinant microorganism is defined as a microorganism which is genetically modified or transformed/transfected with one or more of the nucleotide sequences as defined herein. The presence of the one or more such nucleotide sequences alters the ability of the microorganism to produce a diterpene or diterpene glycoside, in particular steviol or steviol glycoside. A microorganism that is not transformed/transfected or genetically modified, is not a recombinant microorganism and does typically not comprise one or more of the nucleotide sequences enabling the cell to produce a diterpene or diterpene glycoside. Hence, a non-transformed/non-transfected microorganism is typically a microorganism that does not naturally produce a diterpene, although a microorganism which naturally produces a diterpene or diterpene glycoside and which has been modified, as described herein for example (and which thus has an altered ability to produce a diterpene/diterpene gylcoside), is considered a recombinant microorganism.

Sequence identity is herein defined as a relationship between two or more amino acid (polypeptide or protein) sequences or two or more nucleic acid (polynucleotide) sequences, as determined by comparing the sequences. Usually, sequence identities or similarities are compared over the whole length of the sequences compared. In the art, “identity” also means the degree of sequence relatedness between amino acid or nucleic acid sequences, as the case may be, as determined by the match between strings of such sequences. “Identity” and “similarity” can be readily calculated by various methods, known to those skilled in the art. Preferred methods to determine identity are designed to give the largest match between the sequences tested. Typically then, identities and similarities are calculated over the entire length of the sequences being compared. Methods to determine identity and similarity are codified in publicly available computer programs. Preferred computer program methods to determine identity and similarity between two sequences include e.g. the BestFit, BLASTP, BLASTN, and FASTA (Altschul, S. F. et al., J. Mol. Biol. 215:403-410 (1990), publicly available from NCBI and other sources (BLAST Manual, Altschul, S., et al., NCBI NLM NIH Bethesda, Md. 20894). Preferred parameters for amino acid sequences comparison using BLASTP are gap open 10.0, gap extend 0.5, Blosum 62 matrix. Preferred parameters for nucleic acid sequences comparison using BLASTP are gap open 10.0, gap extend 0.5, DNA full matrix (DNA identity matrix).

Nucleotide sequences encoding the enzymes expressed in the cells described herein may also be defined by their capability to hybridize with the nucleotide sequences of SEQ ID NO.'s 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81 or 84 it any other sequence mentioned herein respectively, under moderate, or preferably under stringent hybridisation conditions. Stringent hybridisation conditions are herein defined as conditions that allow a nucleic acid sequence of at least about 25, preferably about 50 nucleotides, 75 or 100 and most preferably of about 200 or more nucleotides, to hybridise at a temperature of about 65° C. in a solution comprising about 1 M salt, preferably 6×SSC or any other solution having a comparable ionic strength, and washing at 65° C. in a solution comprising about 0.1 M salt, or less, preferably 0.2×SSC or any other solution having a comparable ionic strength. Preferably, the hybridisation is performed overnight, i.e. at least for 10 hours and preferably washing is performed for at least one hour with at least two changes of the washing solution. These conditions will usually allow the specific hybridisation of sequences having about 90% or more sequence identity.

Moderate conditions are herein defined as conditions that allow a nucleic acid sequences of at least 50 nucleotides, preferably of about 200 or more nucleotides, to hybridise at a temperature of about 45° C. in a solution comprising about 1 M salt, preferably 6×SSC or any other solution having a comparable ionic strength, and washing at room temperature in a solution comprising about 1 M salt, preferably 6×SSC or any other solution having a comparable ionic strength. Preferably, the hybridisation is performed overnight, i.e. at least for 10 hours, and preferably washing is performed for at least one hour with at least two changes of the washing solution. These conditions will usually allow the specific hybridisation of sequences having up to 50% sequence identity. The person skilled in the art will be able to modify these hybridisation conditions in order to specifically identify sequences varying in identity between 50% and 90%.

The nucleotide sequences encoding an ent-copalyl pyrophosphate synthase; ent-Kaurene synthase; ent-Kaurene oxidase; kaurenoic acid 13-hydroxylase; UGT; hydroxymethylglutaryl-CoA reductase, farnesyl-pyrophosphate synthetase; geranylgeranyl diphosphate synthase; NADPH-cytochrome p450 reductase, may be from prokaryotic or eukaryotic origin.

A nucleotide sequence encoding an ent-copalyl pyrophosphate synthase may for instance comprise a sequence as set out in SEQ ID. NO: 1, 3, 5, 7, 17, 19, 59, 61, 141, 142, 151, 152, 153, 154, 159, 160, 182 or 184.

A nucleotide sequence encoding an ent-Kaurene synthase may for instance comprise a sequence as set out in SEQ ID. NO: 9, 11, 13, 15, 17, 19, 63, 65, 143, 144, 155, 156, 157, 158, 159, 160, 183 or 184.

A nucleotide sequence encoding an ent-Kaurene oxidase may for instance comprise a sequence as set out in SEQ ID. NO: 21, 23, 25, 67, 85, 145, 161, 162, 163, 180 or 186. A preferred KO is the polypeptide encoded by the nucleic acid set out in SEQ ID NO: 85.

A nucleotide sequence encoding a kaurenoic acid 13-hydroxylase may for instance comprise a sequence as set out in SEQ ID. NO: 27, 29, 31, 33, 69, 89, 91, 93, 95, 97, 146, 164, 165, 166, 167 or 185. A preferred KAH sequence is the polypeptide encoded by the nucleic acid set out in SEQ ID NO: 33.

A suitable recombinant microorganism may express a combination of the polypeptides encoded by SEQ ID NO: 85 and SEQ ID NO: 33 or a variant of either thereof as herein described. A preferred recombinant microorganism may express the combination of sequences set out in Table 8 (in combination with any UGT2, but in particular that encoded by SEQ ID NO: 87).

A nucleotide sequence encoding a UGT may for instance comprise a sequence as set out in SEQ ID. NO: 35, 37, 39, 41, 43, 45, 47, 49, 51, 71, 73, 75, 168, 169, 170, 171, 172, 173, 174, 175, 176, 147, 148, 149, 87, 181, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 189, 190, 191 or 192.

A nucleotide sequence encoding a hydroxymethylglutaryl-CoA reductase may for instance comprise a sequence as set out in SEQ ID. NO: 79.

A nucleotide sequence encoding a farnesyl-pyrophosphate synthetase may for instance comprise a sequence as set out in SEQ ID. NO: 81.

A nucleotide sequence encoding a geranylgeranyl diphosphate synthase may for instance comprise a sequence as set out in SEQ ID. NO:83.

A nucleotide sequence encoding a NADPH-cytochrome p450 reductase may for instance comprise a sequence as set out in SEQ ID. NO: 53, 55, 57 or 77.

In the case of the UGT sequences, combinations of at least one from each of: (i) SEQ ID NOs: 35, 37, 168, 169, 71, 147 or 189; (ii) SEQ ID NOs: 87, 99, 101, 103, 105, 107, 109, 111, 181 or 192; (iii) SEQ ID NOs: 39, 41, 43, 45, 47, 170, 171, 172, 173, 174, 73, 148 or 190; and (iv) SEQ ID NOs: 49, 51, 175, 176, 75, 149 or 191 may be preferred. Typically, at least one UGT from group (i) may be used. If at least one UGT from group (iii) is used, generally at least one UGT from group (i) is also used. If at least one UGT from group (iv) is used, generally at least one UGT from group (i) and at least one UGT from group (iii) is used. Typically, at least one UGT form group (ii) is used.

A sequence which has at least about 10%, about 15%, about 20%, preferably at least about 25%, about 30%, about 40%, about 50%, about 55%, about 60%, about 65%, about 70%, about 75%, about 80%, about 85%, about 90%, about 95%, about 96%, about 97%, about 98%, or about 99% sequence identity with a sequence as mentioned may be used in the invention.

To increase the likelihood that the introduced enzymes are expressed in active form in a recombinant microorganism, the corresponding encoding nucleotide sequence may be adapted to optimise its codon usage to that of the chosen eukaryote host cell. The adaptiveness of the nucleotide sequences encoding the enzymes to the codon usage of the chosen host cell may be expressed as codon adaptation index (CAI). The codon adaptation index is herein defined as a measurement of the relative adaptiveness of the codon usage of a gene towards the codon usage of highly expressed genes. The relative adaptiveness (w) of each codon is the ratio of the usage of each codon, to that of the most abundant codon for the same amino acid. The CAI index is defined as the geometric mean of these relative adaptiveness values. Non-synonymous codons and termination codons (dependent on genetic code) are excluded. CAI values range from 0 to 1, with higher values indicating a higher proportion of the most abundant codons (see Sharp and Li, 1987, Nucleic Acids Research 15: 1281-1295; also see: Jansen et al., 2003, Nucleic Acids Res. 31(8):2242-51). An adapted nucleotide sequence preferably has a CAI of at least 0.2, 0.3, 0.4, 0.5, 0.6 or 0.7.

In a preferred embodiment the recombinant is genetically modified with (a) nucleotide sequence(s) which is (are) adapted to the codon usage of the eukaryotic cell using codon pair optimisation technology as disclosed in PCT/EP2007/05594. Codon-pair optimisation is a method for producing a polypeptide in a host cell, wherein the nucleotide sequences encoding the polypeptide have been modified with respect to their codon-usage, in particular the codon-pairs that are used, to obtain improved expression of the nucleotide sequence encoding the polypeptide and/or improved production of the polypeptide. Codon pairs are defined as a set of two subsequent triplets (codons) in a coding sequence.

Further improvement of the activity of the enzymes in vivo in a recombinant microorganism, can be obtained by well-known methods like error prone PCR or directed evolution. A preferred method of directed evolution is described in WO03010183 and WO03010311.

A suitable recombinant microorganism may be any suitable host cell from microbial origin. Preferably, the host cell is a yeast or a filamentous fungus. More preferably, the host cell belongs to one of the genera Saccharomyces, Aspergillus, Penicillium, Pichia, Kluyveromyces, Yarrowia, Candida, Hansenula, Humicola, Torulaspora, Trichosporon, Brettanomyces, Pachysolen or Yamadazyma or Zygosaccharomyces.

A more preferred microorganism belongs to the species Aspergillus niger, Penicillium chrysogenum, Pichia stipidis, Kluyveromyces marxianus, K. lactis, K. thermotolerans, Yarrowia lipolytica, Candida sonorensis, C. glabrata, Hansenula polymorpha, Torulaspora delbrueckii, Brettanomyces bruxellensis, Zygosaccharomyces bailii, Saccharomyces uvarum, Saccharomyces bayanus or Saccharomyces cerevisiae species. Preferably, the eukaryotic cell is a Saccharomyces cerevisiae.

A recombinant yeast cell may be modified so that the ERG9 gene is down-regulated and or the ERG5/ERG6 genes are deleted. Corresponding genes may be modified in this way in other microorganisms.

Such a microorganism may be transformed, whereby the nucleotide sequence(s) with which the microorganism is transformed confer(s) on the cell the ability to produce a diterpene or glycoside thereof.

A preferred suitable recombinant microorganism is a yeast, such as a Saccharomyces cerevisiae or Yarrowia lipolytica cell. A recombinant microorganism, such as a recombinant Saccharomyces cerevisiae cell or Yarrowia lipolytica cell may comprise one or more nucleotide sequence(s) from each of the following groups;

(i) SEQ ID. NO: 1, 3, 5, 7, 17, 19, 59, 61, 141, 142, 152, 153, 154, 159, 160, 182 or 184.

(ii) SEQ ID. NO: 9, 11, 13, 15, 17, 19, 63, 65, 143, 144, 155, 156, 157, 158, 159, 160, 183 or 184.

(iii) SEQ ID. NO: 21, 23, 25, 67 85, 145, 161, 162, 163, 180 or 186.

(iv) SEQ ID. NO: 27, 29, 31, 33, 69, 89, 91, 93, 95, 97, 146, 164, 165, 166, 167 or 185.

Such a microorganism will typically also comprise one or more nucleotide sequence(s) as set out in SEQ ID. NO: 53, 55, 57 or 77.

Such a microorganism may also comprise one or more nucleotide sequences as set out in 35, 37, 39, 41, 43, 45, 47, 49, 51, 71, 73, 75, 168, 169, 170, 171, 172, 173, 174, 175, 176, 147, 148, 149, 87, 181, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 189, 190, 191 or 192. In the case of these sequences, combinations of at least one from each of (i) SEQ ID NOs: 35, 37, 168, 169, 71, 147 or 189; (ii) SEQ ID NOs: 87, 99, 101, 103, 105, 107, 109, 111, 181 or 192; (iii) SEQ ID NOs: 39, 41, 43, 45, 47, 170, 171, 172, 173, 174, 73, 148 or 190; and (iv) SEQ ID NOs: 49, 51, 175, 176, 75, 149 or 191 may be preferred. Typically, at least one UGT from group (i) may be used. If at least one UGT from group (iii) is used, generally at least one UGT from group (i) is also used. If at least one UGT from group (iv) is used, generally at least one UGT from group (i) and at least one UGT from group (iii) is used. Typically, at least one UGT form group (ii) is used.

Such a microorganism may also comprise the following nucleotide sequences: SEQ ID. NO: 79; SEQ ID. NO: 81; and SEQ ID. NO: 83.

For each sequence set out above (or any sequence mentioned herein), a variant having at least about 15%, preferably at least about 20, about 25, about 30, about 40, about 50, about 55, about 60, about 65, about 70, about 75, about 80, about 85, about 90, about 95, about 96, about 97, about 98, or about 99%, sequence identity with the stated sequence may be used.

The nucleotide sequences encoding the ent-copalyl pyrophosphate synthase, ent-Kaurene synthase, ent-Kaurene oxidase, kaurenoic acid 13-hydroxylase, UGTs, hydroxymethylglutaryl-CoA reductase, farnesyl-pyrophosphate synthetase, geranylgeranyl diphosphate synthase and NADPH-cytochrome p450 reductase may be ligated into one or more nucleic acid constructs to facilitate the transformation of the microorganism.

A nucleic acid construct may be a plasmid carrying the genes encoding enzymes of the diterpene, e.g. steviol/steviol glycoside, pathway as described above, or a nucleic acid construct may comprise two or three plasmids carrying each three or two genes, respectively, encoding the enzymes of the diterpene pathway distributed in any appropriate way.

Any suitable plasmid may be used, for instance a low copy plasmid or a high copy plasmid.

It may be possible that the enzymes selected from the group consisting of ent-copalyl pyrophosphate synthase, ent-Kaurene synthase, ent-Kaurene oxidase, and kaurenoic acid 13-hydroxylase, UGTs, hydroxymethylglutaryl-CoA reductase, farnesyl-pyrophosphate synthetase, geranylgeranyl diphosphate synthase and NADPH-cytochrome p450 reductase are native to the host microorganism and that transformation with one or more of the nucleotide sequences encoding these enzymes may not be required to confer the host cell the ability to produce a diterpene or diterpene glycosidase. Further improvement of diterpene/diterpene glycosidase production by the host microorganism may be obtained by classical strain improvement.

The nucleic acid construct may be maintained episomally and thus comprise a sequence for autonomous replication, such as an autosomal replication sequence sequence. If the host cell is of fungal origin, a suitable episomal nucleic acid construct may e.g. be based on the yeast 2μ or pKD1 plasmids (Gleer et al., 1991, Biotechnology 9: 968-975), or the AMA plasmids (Fierro et al., 1995, Curr Genet. 29:482-489).

Alternatively, each nucleic acid construct may be integrated in one or more copies into the genome of the host cell. Integration into the host cell's genome may occur at random by non-homologous recombination but preferably the nucleic acid construct may be integrated into the host cell's genome by homologous recombination as is well known in the art (see e.g. WO90/14423, EP-A-0481008, EP-A-0635 574 and U.S. Pat. No. 6,265,186).

Optionally, a selectable marker may be present in the nucleic acid construct. As used herein, the term “marker” refers to a gene encoding a trait or a phenotype which permits the selection of, or the screening for, a microorganism containing the marker. The marker gene may be an antibiotic resistance gene whereby the appropriate antibiotic can be used to select for transformed cells from among cells that are not transformed. Alternatively or also, non-antibiotic resistance markers are used, such as auxotrophic markers (URA3, TRP1, LEU2). The host cells transformed with the nucleic acid constructs may be marker gene free. Methods for constructing recombinant marker gene free microbial host cells are disclosed in EP-A-0 635 574 and are based on the use of bidirectional markers. Alternatively, a screenable marker such as Green Fluorescent Protein, lacZ, luciferase, chloramphenicol acetyltransferase, beta-glucuronidase may be incorporated into the nucleic acid constructs allowing for screening for transformed cells. A preferred marker-free method for the introduction of heterologous polynucleotides is described in WO0540186.

In a preferred embodiment, the nucleotide sequences encoding ent-copalyl pyrophosphate synthase, ent-Kaurene synthase, ent-Kaurene oxidase, and kaurenoic acid 13-hydroxylase, UGTs, hydroxymethylglutaryl-CoA reductase, farnesyl-pyrophosphate synthetase, geranylgeranyl diphosphate synthase and NADPH-cytochrome p450 reductase, are each operably linked to a promoter that causes sufficient expression of the corresponding nucleotide sequences in the recombinant microorganism to confer to the cell the ability to produce a diterpene or diterpene glycoside.

As used herein, the term “operably linked” refers to a linkage of polynucleotide elements (or coding sequences or nucleic acid sequence) in a functional relationship. A nucleic acid sequence is “operably linked” when it is placed into a functional relationship with another nucleic acid sequence. For instance, a promoter or enhancer is operably linked to a coding sequence if it affects the transcription of the coding sequence.

As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skilled in the art to act directly or indirectly to regulate the amount of transcription from the promoter. A “constitutive” promoter is a promoter that is active under most environmental and developmental conditions. An “inducible” promoter is a promoter that is active under environmental or developmental regulation.

The promoter that could be used to achieve the expression of the nucleotide sequences coding for an enzyme as defined herein above, may be not native to the nucleotide sequence coding for the enzyme to be expressed, i.e. a promoter that is heterologous to the nucleotide sequence (coding sequence) to which it is operably linked. Preferably, the promoter is homologous, i.e. endogenous to the host cell

Suitable promoters for use in recombinant microorganisms may be GAL7, GAL10, or GAL 1, CYC1, HIS3, ADH1, PGL, PH05, GAPDH, ADC1, TRP1, URA3, LEU2, ENO, TPI, and AOX1. Other suitable promoters include PDC, GPD1, PGK1, TEF1, and TDH.

Any terminator, which is functional in the cell, may be used. Preferred terminators are obtained from natural genes of the host cell. Suitable terminator sequences are well known in the art. Preferably, such terminators are combined with mutations that prevent nonsense mediated mRNA decay in the host cell (see for example: Shirley et al., 2002, Genetics 161:1465-1482).

Nucleotide sequences used may include sequences which target them to desired compartments of the microorganism. For example, in a preferred recombinant microorganism, all nucleotide sequences, except for ent-Kaurene oxidase, kaurenoic acid 13-hydroxylase and NADPH-cytochrome p450 reductase encoding sequences may be targeted to the cytosol. This approach may be used in a yeast cell.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain.

The term “heterologous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous nucleic acids or proteins are not endogenous to the cell into which it is introduced, but have been obtained from another cell or synthetically or recombinantly produced.

Typically, a suitable recombinant microorganism will comprise heterologous nucleotide sequences. Alternatively, a recombinant microorganism may comprise entirely homologous sequence which has been modified as set out herein so that the microorganism produces increased amounts of a diterpene and/or diterpene glycoside in comparison to a non-modified version of the same microorganism.

One or more enzymes of the diterpene pathway as described herein may be overexpressed to achieve a sufficient diterpene production by the cell.

There are various means available in the art for overexpression of enzymes in the host cell. In particular, an enzyme may be overexpressed by increasing the copy number of the gene coding for the enzyme in the host cell, e.g. by integrating additional copies of the gene in the host cell's genome.

A preferred recombinant microorganism may be a recombinant microorganism which is naturally capable of producing GGPP.

A suitable recombinant microorganism may be able to grow on any suitable carbon source known in the art and convert it to one or more steviol glycosides. The recombinant microorganism may be able to convert directly plant biomass, celluloses, hemicelluloses, pectines, rhamnose, galactose, fucose, maltose, maltodextrines, ribose, ribulose, or starch, starch derivatives, sucrose, lactose and glycerol. Hence, a preferred host organism expresses enzymes such as cellulases (endocellulases and exocellulases) and hemicellulases (e.g. endo- and exo-xylanases, arabinases) necessary for the conversion of cellulose into glucose monomers and hemicellulose into xylose and arabinose monomers, pectinases able to convert pectines into glucuronic acid and galacturonic acid or amylases to convert starch into glucose monomers. Preferably, the host cell is able to convert a carbon source selected from the group consisting of glucose, xylose, arabinose, sucrose, lactose and glycerol. The host cell may for instance be a eukaryotic host cell as described in WO03/062430, WO06/009434, EP1499708B1, WO2006096130 or WO04/099381.

A recombinant microorganism as described above may be used in a process for the production of a steviol glycoside, which method comprises fermenting a transformed a suitable recombinant microorganism (as described herein) in a suitable fermentation medium, and optionally recovering the diterpene and/or diterpene glycoside.

The fermentation medium used in the process for the production of a diterpene or diterpene glycoside may be any suitable fermentation medium which allows growth of a particular eukaryotic host cell. The essential elements of the fermentation medium are known to the person skilled in the art and may be adapted to the host cell selected.

Preferably, the fermentation medium comprises a carbon source selected from the group consisting of plant biomass, celluloses, hemicelluloses, pectines, rhamnose, galactose, fucose, fructose, maltose, maltodextrines, ribose, ribulose, or starch, starch derivatives, sucrose, lactose, fatty acids, triglycerides and glycerol. Preferably, the fermentation medium also comprises a nitrogen source such as ureum, or an ammonium salt such as ammonium sulphate, ammonium chloride, ammoniumnitrate or ammonium phosphate.

A suitable fermentation process may be carried out in batch, fed-batch or continuous mode. A separate hydrolysis and fermentation (SHF) process or a simultaneous saccharification and fermentation (SSF) process may also be applied. A combination of these fermentation process modes may also be possible for optimal productivity. A SSF process may be particularly attractive if starch, cellulose, hemicelluose or pectin is used as a carbon source in the fermentation process, where it may be necessary to add hydrolytic enzymes, such as cellulases, hemicellulases or pectinases to hydrolyse the substrate.

The recombinant microorganism used in the process for the preparation of a steviol glycoside may be any suitable microorganism as defined herein above. It may be advantageous to use a recombinant eukaryotic microorganism as described herein in the process for the production of a diterpene or diterpene glycoside, because most eukaryotic cells do not require sterile conditions for propagation and are insensitive to bacteriophage infections. In addition, eukaryotic host cells may be grown at low pH to prevent bacterial contamination.

The recombinant microorganism may be a facultative anaerobic microorganism. A facultative anaerobic microorganism can be propagated aerobically to a high cell concentration. This anaerobic phase can then be carried out at high cell density which reduces the fermentation volume required substantially, and may minimize the risk of contamination with aerobic microorganisms.

The fermentation process for the production of a steviol glycoside may be an aerobic or an anaerobic fermentation process.

An anaerobic fermentation process may be herein defined as a fermentation process run in the absence of oxygen or in which substantially no oxygen is consumed, preferably less than 5, 2.5 or 1 mmol/L/h, and wherein organic molecules serve as both electron donor and electron acceptors. The fermentation process may also first be run under aerobic conditions and subsequently under anaerobic conditions.

The fermentation process may also be run under oxygen-limited, or micro-aerobical, conditions. Alternatively, the fermentation process may first be run under aerobic conditions and subsequently under oxygen-limited conditions. An oxygen-limited fermentation process is a process in which the oxygen consumption is limited by the oxygen transfer from the gas to the liquid. The degree of oxygen limitation is determined by the amount and composition of the ingoing gas flow as well as the actual mixing/mass transfer properties of the fermentation equipment used.

The production of a steviol glycoside in the fermentation process may occur during the growth phase of the host cell, during the stationary (steady state) phase or during both phases. It may be possible to run the fermentation process at different temperatures.

The process for the production of a steviol glycoside may be run at a temperature which is optimal for the recombinant microorganism. The optimum growth temperature may differ for each transformed cell and is known to the person skilled in the art. The optimum temperature might be higher than optimal for wild type organisms to grow the organism efficiently under non-sterile conditions under minimal infection sensitivity and lowest cooling cost. Alternatively, the process may be carried out at a temperature which is not optimal for growth of the recombinant microorganism.

The temperature for growth of the recombinant microorganism in a process for production of a diterpene or diterpene glycoside may be above 20° C., 22° C., 25° C., 28° C., or above 30° C., 35° C., or above 37° C., 40° C., 42° C., and preferably below 45° C. During the production phase of a diterpene or diterpene glycoside however, the optimum temperature might be lower than average in order to optimize biomass stability. The temperature during this phase may be below 45° C., for instance below 42° C., 40° C., 37° C., for instance below 35° C., 30° C., or below 28° C., 25° C., 22° C. or below 20° C. preferably above 15° C.

The process for the production of a steviol glycoside may be carried out at any suitable pH value. If the recombinant microorganism is yeast, the pH in the fermentation medium preferably has a value of below 6, preferably below 5.5, preferably below 5, preferably below 4.5, preferably below 4, preferably below pH 3.5 or below pH 3.0, or below pH 2.5, preferably above pH 2. An advantage of carrying out the fermentation at these low pH values is that growth of contaminant bacteria in the fermentation medium may be prevented.

Such a process may be carried out on an industrial scale.

The product of such a process may be one or more of steviolmonoside, steviolbioside, stevioside or rebaudioside A, rebaudioside B, rebaudioside C, rebaudioside D, rebaudioside E, rebaudioside F, rubusoside, dulcoside A. Preferably, rebaudioside A or rebaudioside D is produced.

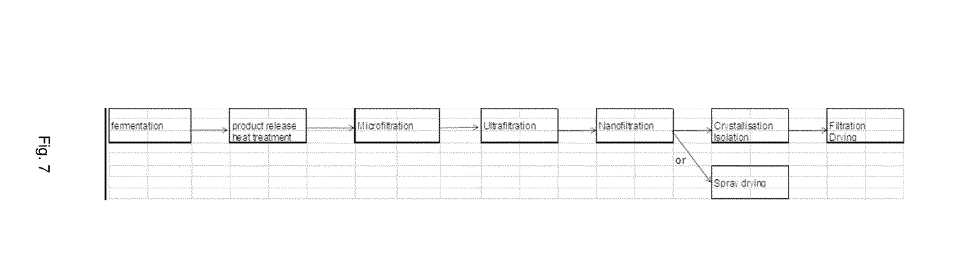

Recovery of the diterpene or diterpene glycoside from the resulting broth may be carried out by known methods in the art, for instance by filtration, crystallization, distillation, vacuum extraction, solvent extraction, or evaporation. In the event that Reb-A is expressed within the microorganism, such cells may need to be treated so as to release Reb-A. FIG. 7 sets out a scheme for recovery of steviol glycosides from a fermentation broth

In the process for the fermentative production of a steviol glycoside, it may be possible to achieve a concentration of above 5 mg/l fermentation broth, preferably above 10 mg/l, preferably above 20 mg/l, preferably above 30 mg/l fermentation broth, preferably above 40 mg/l, more preferably above 50 mg/l, preferably above 60 mg/l, preferably above 70, preferably above 80 mg/l, preferably above 100 mg/l, preferably above 1 g/l, preferably above 5 g/l, preferably above 10 g/l, but usually below 70 g/l in the broth.

As described above, in the event that a diterpene or diterpene glycoside is expressed within the microorganism, such cells may need to be treated so as to release the steviol glycoside.

Steviol glycosides produced as described herein may be blended with one or more further non-calorific or calorific sweeteners. Such blending may be used to improve flavor or temporal profile or stability. A wide range of both non-calorific and calorific sweeteners may be suitable for blending with Reb-A. For example, non-calorific sweeteners such as mogroside, monatin, aspartame, acesulfame salts, cyclamate, sucralose, saccharin salts or erythritol. Calorific sweeteners suitable for blending with Reb-A include sugar alcohols and carbohydrates such as sucrose, glucose, fructose and HFCS. Sweet tasting amino acids such as glycine, alanine or serine may also be used.

Steviol glycosides (containing Rebaudioside-A 95%) may be a white granular material that has a sweet taste. A microbial fermentation process is used for production of this material. Reb-A or steviol glycosides is soluble in water at a level greater than 3000 ppm (>0.3%). This material contains no detectable microbial residues. This material is a Food Additive and Kosher Pareve. The ingredients in this material are derived from recombinant microbial sources. Following are detailed product specifications:

1. Assay (wt/wt %): Greater than or equal to 95% Reb-A (on dry basis)

2. Total Steviol Glycosides (2 t/wt %): Greater than 95% (on dry basis)

3. Stevioside (wt/wt %): 2% (on dry basis) maximum

4. Steviol (wt/wt %): Less than 0.005% (on dry basis)

5. Moisture Content (%) by loss on drying: 6% maximum

6. Optical Rotation: −29 to 37 Degrees

7. pH: 4.5-7.0 (1 g in 100 ml water)

8. Arsenic (as As): 1 mg/kg maximum

9. Lead (as Pb): 1 mg/kg maximum

10. Mercusry (Hb): 1 mg/kg maximum

11. Cadmium (Cd): 1 mg/kg maximum

12. Total Aerobic Plate Count: 1000 CFU/g maximum (CFU=colony forming unit)

13. Total Aerobic Mold count: 1000 CFU/g maximum

14. Total Aerobic Yeast count: 1000 CFU/g maximum

15. Coliform: Less than 10 CFU/g

16. E. coli: less than 3 MPN/g (MPN=most probable number)

17. Residue on Ignition: 1.0% maximum (synonym for Ash)

18. Residual solvents: MeOH <200 ppm; EtOH <5000 ppm

The sweetener composition (i.e. a composition comprises a steviol glycoside such as rebA) and product specifications described herein may be applied for use in any suitable product such as zero calorie, reduced calorie or diabetic beverages and food products with improved taste characteristics. Also it can be used in drinks, foodstuffs, pharmaceuticals, and other products in which sugar cannot be used.

In addition, the sweetener composition can be used as a sweetener not only for drinks, foodstuffs, and other products dedicated for human consumption, but also in animal feed and fodder with improved characteristics.

The examples of products where the sweetener composition can be used as sweetening compound can be as alcoholic beverages such as vodka, wine, beer, liquor, sake, etc; natural juices, refreshing drinks, carbonated soft drinks, diet drinks, zero calorie drinks, reduced calorie drinks and foods, yogurt drinks, instant juices, instant coffee, powdered types of instant beverages, canned products, syrups, fermented soybean paste, soy sauce, vinegar, dressings, mayonnaise, ketchups, curry, soup, instant bouillon, powdered soy sauce, powdered vinegar, types of biscuits, rice biscuit, crackers, bread, chocolates, caramel, candy, chewing gum, jelly, pudding, preserved fruits and vegetables, fresh cream, jam, marmalade, flower paste, powdered milk, ice cream, sorbet, vegetables and fruits packed in bottles, canned and boiled beans, meat and foods boiled in sweetened sauce, agricultural vegetable food products, seafood, ham, sausage, fish ham, fish sausage, fish paste, deep fried fish products, dried seafood products, frozen food products, preserved seaweed, preserved meat, tobacco, medicinal products, and many others. In principal it can have unlimited applications.

The sweetened composition comprises a beverage, non-limiting examples of which include non-carbonated and carbonated beverages such as colas, ginger ales, root beers, ciders, fruit-flavored soft drinks (e.g., citrus-flavored soft drinks such as lemon-lime or orange), powdered soft drinks, and the like; fruit juices originating in fruits or vegetables, fruit juices including squeezed juices or the like, fruit juices containing fruit particles, fruit beverages, fruit juice beverages, beverages containing fruit juices, beverages with fruit flavorings, vegetable juices, juices containing vegetables, and mixed juices containing fruits and vegetables; sport drinks, energy drinks, near water and the like drinks (e.g., water with natural or synthetic flavorants); tea type or favorite type beverages such as coffee, cocoa, black tea, green tea, oolong tea and the like; beverages containing milk components such as milk beverages, coffee containing milk components, cafe au lait, milk tea, fruit milk beverages, drinkable yogurt, lactic acid bacteria beverages or the like; and dairy products.

The sweetener composition described herein may be incorporated as a high intensity natural sweetener in foodstuffs, beverages, pharmaceutical compositions, cosmetics, chewing gums, table top products, cereals, dairy products, toothpastes and other oral cavity compositions, etc.

In addition, the sweetener composition can be used as a sweetener not only for drinks, foodstuffs, and other products dedicated for human consumption, but also in animal feed and fodder with improved characteristics

During the manufacturing of foodstuffs, drinks, pharmaceuticals, cosmetics, table top products, chewing gum the conventional methods such as mixing, kneading, dissolution, pickling, permeation, percolation, sprinkling, atomizing, infusing and other methods can be used.

The sweetener composition can be used in dry or liquid forms. It can be added before or after heat treatment of food products. The amount of the sweetener depends on the purpose of usage. It can be added alone or in the combination with other compounds.

The sweetener composition may be employed as the sole sweetener, or it may be used together with other naturally occurring high intensity sweeteners.

The phrase “natural high intensity sweeteners”, as used herein, refers to any compositions which are found in nature and which have sweetness potency higher than sucrose, fructose, or glucose.

Non-limiting examples of natural high intensity sweeteners include Stevioside, Rebaudioside A, Rebaudioside B, Rebaudioside C, Rebaudioside E, Rebaudioside F, Rebaudioside M, Rebaudioside X, Steviolbioside, Dulcoside A, Rubusoside, mogrosides, brazzein, glycyrrhizic acid and its salts, thaumatin, perillartine, pernandulcin, mukuroziosides, baiyunoside, phlomisoside-I, dimethyl-hexahydrofluorene-dicarboxylic acid, abrusosides, periandrin, carnosiflosides, cyclocarioside, pterocaryosides, polypodoside A, brazilin, hernandulcin, phillodulcin, glycyphyllin, phlorizin, trilobatin, dihydroflavonol, dihydroquercetin-3-acetate, neoastilibin, ira″5-cinnamaldehyde, monatin and its salts, selligueain A, hematoxylin, monellin, osladin, pterocaryoside A, pterocaryoside B, mabinlin, pentadin, miraculin, curculin, neoculin, chlorogenic acid, cynarin, Luo Han Guo sweetener, siamenoside and alike, and combinations thereof.

The sweetener composition may be used together with synthetic or artificial high intensity sweeteners. The phrase “synthetic” or “artificial high intensity sweeteners”, as used herein, refers to any compositions which are not found in nature and which have—sweetness potency higher than sucrose, fructose, or glucose. Non-limiting examples of synthetic or artificial high intensity sweeteners include sucralose, potassium acesulfame, aspartame, alitame, saccharin, neohesperidin dihydrochalcone, cyclamate, neotame, dulcin, suosan, N—[N-[3-(3-hydroxy-4-methoxyphenyl)propyl]-L-a-aspartyl]-L-phenylalanine 1-methyl ester, N—[N-[3-(3-hydroxy-4-methoxyphenyl)-3-methylbutyl]-L- -asparty 1]-L-phenylalanine 1-methyl ester, N—[N-[3-(3-methoxy-4-hydroxyphenyl)propyl]-L-a-aspartyl]-L-phenylalanine 1-methyl ester, salts thereof, and the like, and combinations thereof.

In one embodiment rebA can be used in the combination with natural sweetener suppressors such as gymnemic acid, hodulcin, ziziphin, lactisole, and the like.

The sweetener composition can be combined with various umami taste enhancers.

The sweetener composition can be formulated with amino acids including, but not limited to, aspartic acid, arginine, glycine, glutamic acid, proline, threonine, theanine, cysteine, cystine, alanine, valine, tyrosine, leucine, isoleucine, asparagine, serine, lysine, histidine, ornithine, methionine, carnitine, aminobutyric acid (alpha-, beta-, or gamma-isomers), glutamine, hydroxyproline, taurine, norvaline, sarcosine, and their salt forms such as sodium or potassium salts or acid salts. The amino acid additives also may be in the D- or L-configuration and in the mono-, di-, or tri-form of the same or different amino acids. Additionally, the amino acids may be [alpha]-, [beta]-, y˜, [delta]-, and ̂-isomers if appropriate. Combinations of the foregoing amino acids and their corresponding salts (e.g., sodium, potassium, calcium, magnesium salts or other alkali or alkaline earth metal salts thereof, or acid salts) also are suitable additives. The amino acids may be natural or synthetic. The amino acids also may be modified. Modified amino acids refers to any amino acid wherein at least one atom has been added, removed, substituted, or combinations thereof (e.g., N-alkyl amino acid, N-acyl amino acid, or N-methyl amino acid). Non-limiting examples of modified amino acids include amino acid derivatives such as trimethyl glycine, N-methyl-glycine, and N-methyl-alanine. As used herein, amino acids encompass both modified and unmodified amino acids. As used herein, modified amino acid also may encompass peptides and polypeptides (e.g., dipeptides, tripeptides, tetrapeptides, and pentapeptides) such as glutathione and L-alanyl-L-glutamine.

The sweetener composition may be formulated with polyamino acid additives include poly-L-aspartic acid, poly-L-lysine (e.g., poly-L-a-lysine or poly-L-̂-lysine), poly-L-ornithine (e.g., poly-L- -ornithine or poly-L-f-ornithine), poly-L-arginine, other polymeric forms of amino acids, and salt forms thereof (e.g., magnesium, calcium, potassium, or sodium salts such as L-glutamic acid mono sodium salt). The polyamino acid additives also may be in the D- or L-configuration. Additionally, the polyamino acids may be a -, [beta]-, [gamma]-, [delta]-, and [epsilon]-isomers if appropriate. Combinations of the foregoing polyamino acids and their corresponding salts (e.g., sodium, potassium, calcium, magnesium salts or other alkali or alkaline earth metal salts thereof or acid salts) also are suitable sweet taste improving additives in embodiments of this invention. The polyamino acids described herein also may comprise co-polymers of different amino acids. The polyamino acids may be natural or synthetic. The polyamino acids also may be modified, such that at least one atom has been added, removed, substituted, or combinations thereof (e.g., N-alkyl polyamino acid or N-acyl polyamino acid). As used herein, polyamino acids encompass both modified and unmodified polyamino acids. In accordance with particular embodiments, modified polyamino acids include, but are not limited to polyamino acids of various molecular weights (MW), such as poly-L-a-lysine with a MW of 1,500, MW of 6,000, MW of 25,200, MW of 63,000, M W of 83,000, or M W of 300,000.

The sweetener composition can be combined with polyols or sugar alcohols. The term “polyol” refers to a molecule that contains more than one hydroxyl group. A polyol may be a diol, triol, or a tetraol which contain 2, 3, and 4 hydroxyl groups, respectively. A polyol also may contain more than four hydroxyl groups, such as a pentaol, hexaol, heptaol, or the like, which contain 5, 6, or 7 hydroxyl groups, respectively. Additionally, a polyol also may be a sugar alcohol, polyhydric alcohol, or polyalcohol which is a reduced form of carbohydrate, wherein the carbonyl group (aldehyde or ketone, reducing sugar) has been reduced to a primary or secondary hydroxyl group.

Non-limiting examples of polyols include erythritol, maltitol, mannitol, sorbitol, lactitol, xylitol, inositol, isomalt, propylene glycol, glycerol, threitol, galactitol, hydrogenated isomaltulose, reduced isomalto-oligosaccharides, reduced xylo-oligosaccharides, reduced gentio-oligosaccharides, reduced maltose syrup, reduced glucose syrup, hydrogenated starch hydrolyzates, polyglycitols and sugar alcohols or any other carbohydrates capable of being reduced which do not adversely affect the taste of the sweetener composition, and combinations thereof.

In one particular embodiment rebA can be combined with reduced calorie sweeteners such as D-tagatose, L-sugars, L-sorbose, L-arabinose, and others and combinations thereof.

The sweetener composition can be combined with various carbohydrates. The term “carbohydrate” generally refers to aldehyde or ketone compounds substituted with multiple hydroxyl groups, of the general formula (CH20)m wherein “n” is 3-30, as well as their oligomers and polymers. The carbohydrates of the present invention can, in addition, be substituted or deoxygenated at one or more positions. Carbohydrates, as used herein, encompass unmodified carbohydrates, carbohydrate derivatives, substituted carbohydrates, and modified carbohydrates. As used herein, the phrases “carbohydrate derivatives”, “substituted carbohydrate”, and “modified carbohydrates” are synonymous. Modified carbohydrate means any carbohydrate wherein at least one atom has been added, removed, substituted, or combinations thereof. Thus, carbohydrate derivatives or substituted carbohydrates include substituted and unsubstituted monosaccharides, disaccharides, oligosaccharides, and polysaccharides. The carbohydrate derivatives or substituted carbohydrates optionally can be deoxygenated at any corresponding C-position, and/or substituted with one or more moieties such as hydrogen, halogen, haloalkyl, carboxyl, acyl, acyloxy, amino, amido, carboxyl derivatives, alkylamino, dialkylamino, arylamino, alkoxy, aryloxy, nitro, cyano, sulfo, mercapto, imino, sulfonyl, sulfenyl, sulfinyl, sulfamoyl, carboalkoxy, carboxamido, phosphonyl, phosphinyl, phosphoryl, phosphino, thioester, thioether, oximino, hydrazino, carbamyl, phospho, phosphonato, or any other viable functional group provided the carbohydrate derivative or substituted carbohydrate functions to improve the sweet taste of the sweetener composition.

Non-limiting examples of carbohydrates in embodiments of this invention include tagatose, trehalose, galactose, rhamnose, various cyclodextrins, cyclic oligosaccharides, various types of maltodextrins, dextran, sucrose, glucose, ribulose, fructose, threose, arabinose, xylose, lyxose, allose, altrose, mannose, idose, lactose, maltose, invert sugar, isotrehalose, neotrehalose, isomaltulose, erythrose, deoxyribose, gulose, idose, talose, erythrulose, xylulose, psicose, turanose, cellobiose, amylopectin, glucosamine, mannosamine, fucose, glucuronic acid, gluconic acid, glucono-lactone, abequose, galactosamine, beet oligosaccharides, isomalto-oligosaccharides (isomaltose, isomaltotriose, panose and the like), xylo-oligosaccharides (xylotriose, xylobiose and the like), xylo-terminated oligosaccharides, gentio-oligosaccharides (gentiobiose, gentiotriose, gentiotetraose and the like), sorbose, nigero-oligosaccharides, palatinose oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), starch, inulin, inulo-oligosaccharides, lactulose, melibiose, raffinose, ribose, isomerized liquid sugars such as high fructose corn syrups, coupling sugars, and soybean oligosaccharides. Additionally, the carbohydrates as used herein may be in either the D- or L-configuration. In the formulations any combinations of the compounds can be used.

In a particular embodiment rebA may be formulated with sugar acids which is include, but are not limited to, aldonic, uronic, aldaric, alginic, gluconic, glucuronic, glucaric, galactaric, galacturonic, and their salts (e.g., sodium, potassium, calcium, magnesium salts or other physiologically acceptable salts), and combinations thereof.

The sweetener composition can be used in the combination with various physiologically active substances or functional ingredients. Functional ingredients generally are classified into categories such as carotenoids, dietary fiber, fatty acids, saponins, antioxidants, nutraceuticals, flavonoids, isothiocyanates, phenols, plant sterols and stanols (phytosterols and phytostanols); polyols; prebiotics, probiotics; phytoestrogens; soy protein; sulfides/thiols; amino acids; proteins; vitamins; and minerals. Functional ingredients also may be classified based on their health benefits, such as cardiovascular, cholesterol-reducing, and anti-inflammatory.

The sweetener composition may include a flavoring agent which may be natural or artificial origin. As used herein, unless otherwise indicated, the term “flavor” means any food-grade material that may be added to the present compositions to provide a desired flavor to a foodstuff. The flavors useful in the present invention include, for example, an essential oil, such as an oil derived from a plant or a fruit, peppermint oil, spearmint oil, other mint oils, clove oil, cinnamon oil, oil of wintergreen, bay, thyme, cedar leaf, nutmeg, allspice, sage, mace, and almonds. The flavoring agent may be a plant extract or a fruit essence such as apple, banana, watermelon, pear, peach, grape, strawberry, raspberry, cherry, plum, pineapple, apricot, and mixtures thereof. The flavoring agent may be a citrus flavor, such as an extract, essence, or oil of lemon, lime, orange, tangerine, grapefruit, citron, or kumquat. Flavors useful in the present invention also can include cream, hazelnut, vanilla, chocolate, cinnamon, pecan, lemon, lime, raspberry, peach, mango, vanillin, butter, butterscotch, tea, orange, tangerine, caramel, strawberry, banana, grape, plum, cherry, blueberry, pineapple, elderberry, watermelon, bubblegum, cantaloupe, guava, kiwi, papaya, coconut, mint, spearmint, derivatives, and combinations thereof.

The sweetener composition may include an aroma component. As used herein, unless otherwise indicated, the term “aroma component” means any food-grade volatile substance that may be employed to produce a desired scent, for example, when mixed with a foodstuff. Aromas useful in the present invention include, for example, essential oils (citrus oil), expressed oils (orange oil), distilled oils (rose oil), extracts (fruits), anethole (liquorice, anise seed, ouzo, fennel), anisole (anise seed), benzaldehyde (marzipan, almond), benzyl alcohol (marzipan, almond), camphor (cinnamomum camphora), cinnamaldehyde (cinnamon), citral (citronella oil, lemon oil), d-limonene (orange) ethyl butanoate (pineapple), eugenol (clove oil), furaneol (strawberry), furfural (caramel), linalool (coriander, rose wood), menthol (peppermint), methyl butanoate (apple, pineapple), methyl salicylate (oil of wintergreen), neral (orange flowers), nerolin (orange flowers), pentyl butanoate (pear, apricot), pentyl pentanoate (apple, pineapple), sotolon (maple syrup, curry, fennugreek), strawberry ketone (strawberry), substituted pyrazines, e.g., 2-ethoxy-3-isopropylpyrazine; 2-methoxy-3-sec-butylpyrazine; and 2-methoxy-3-methylpyrazine (toasted seeds of fenugreek, cumin, and coriander), thujone (juniper, common sage, Nootka cypress, and wormwood), thymol (camphor-like), trimethylamine (fish), vanillin (vanilla), and combinations thereof. Preferred aroma components according to the present invention include, essential oils (citrus oil), expressed oils (orange oil), distilled oils (rose oil), extracts (fruits), benzaldehyde, d-limonene, furfural, menthol, methyl butanoate, pentyl butanoate, salts, derivatives, and combinations thereof.

The sweetener composition can comprise a nucleotide additive for use in embodiments of this invention. They include, but are not limited to, inosine monophosphate, guanosine monophosphate, adenosine monophosphate, cytosine monophosphate, uracil monophosphate, inosine diphosphate, guanosine diphosphate, adenosine diphosphate, cytosine diphosphate, uracil diphosphate, inosine triphosphate, guanosine triphosphate, adenosine triphosphate, cytosine triphosphate, uracil triphosphate, and their alkali or alkaline earth metal salts, and combinations thereof. The nucleotides described herein also may comprise nucleotide-related additives such as nucleosides or nucleic acid bases (e.g., guanine, cytosine, adenine, thymine, uracil).

The sweetener composition can comprise an organic acid additive. Organic acids are compounds which comprises a —COOH moiety. Suitable organic acid additives for use in embodiments of this invention include, but are not limited to, C2-C30 carboxylic acids, substituted hydroxy 1C1-C30 carboxylic acids, benzoic acid, substituted benzoic acids (e.g. 2,4-dihydroxybenzoic acid), substituted cinnamic acids, hydroxyacids, substituted hydroxybenzoic acids, substituted cyclohexyl carboxylic acids, tannic acid, lactic acid, tartaric acid, citric acid, gluconic acid, glucoheptonic acids, adipic acid, hydroxycitric acid, malic acid, fruitaric acid (a blend of malic, fumaric, and tartaric acids), fumaric acid, maleic acid, succinic acid, chlorogenic acid, salicylic acid, creatine, glucosamine hydrochloride, glucono delta lactone, caffeic acid, bile acids, acetic acid, ascorbic acid, alginic acid, crythorbic acid, polyglutamic acid, and their alkali or alkaline earth metal salt derivatives thereof. In addition, the organic acid additives also may be in either the D- or L-configuration.

The sweetener composition can comprise an organic acid salt additive. They include, but are not limited to, sodium, calcium, potassium, and magnesium salts of all organic acids, such as salts of citric acid, malic acid, tartaric acid, flunaric acid, lactic acid (e.g., sodium lactate), alginic acid (e.g., sodium alginate), ascorbic acid (e.g., sodium ascorbate), benzoic acid (e.g., sodium benzoate or potassium benzoate), and adipic acid. The examples of the sweet taste improving organic acid salt additives described optionally may be substituted with one or more of the following moiety selected from the group consisting of hydrogen, alkyl, alkenyl, alkynyl, halo, haloalkyl, carboxyl, acyl, acyloxy, amino, amido, carboxyl derivatives, alkylamino, dialkylamino, arylamino, alkoxy, aryloxy, nitro, cyano, sulfo, thiol, imine, sulfonyl, sulfenyl, sulfinyl, sulfamyl, carboxalkoxy, carboxamido, phosphonyl, phosphinyl, phosphoryl, phosphino, thioester, thioether, anhydride, oximino, hydrazino, carbamyl, phospho, phosphonato, and any other viable functional group, provided the substituted organic acid salt additive functions to improve the sweet taste of the sweetener composition.

The compositions with rebA can comprise an inorganic acid additive for use in embodiments of this invention. They include, but are not limited to, phosphoric acid, phosphorous acid, polyphosphoric acid, hydrochloric acid, sulfuric acid, carbonic acid, sodium dihydrogen phosphate, and their corresponding alkali or alkaline earth metal salts thereof (e.g., inositol hexaphosphate Mg Ca).

The sweetener composition can comprise a bitter compound additive for use in embodiments of this invention, but are not limited to, caffeine, quinine, urea, bitter orange oil, naringin, quassia, and salts thereof.

The sweetener composition can comprise an artificial or natural sweetness enhancers and combinations thereof.

The sweetener composition may include a polymer additives for use in embodiments of this invention, but are not limited to, chitosan, pectin, pectic, pectinic, polyuronic, polygalacturonic acid, starch, food hydrocolloid or crude extracts thereof (e.g., gum acacia Senegal (Fibergum™), gum acacia seyal, carageenan), poly-L-lysine (e.g., poly-L-a-lysine or poly-L-f-lysine), poly-L-ornithine (e.g., poly-L-a-ornithine or poly-L-[epsilon]-ornithine), polyarginine, polypropylene glycol, polyethylene glycol, poly(ethylene glycol methyl ether), polyaspartic acid, polyglutamic acid, polyethyleneimine, alginic acid, sodium alginate, propylene glycol alginate, sodium hexametaphosphate (SHMP) and its salts, and sodium polyethyleneglycolalginate and other cationic and anionic polymers.

The sweetener compositions may include a protein or protein hydrolyzates additives for use in embodiments of this invention, but are not limited to, bovine serum albumin, whey protein (including fractions or concentrates thereof such as 90% instant whey protein isolate, 34% whey protein, 50% hydrolyzed whey protein, and 80% whey protein concentrate), soluble rice protein, soy protein, protein isolates, protein hydrolyzates, reaction products of protein hydrolyzates, glycoproteins, and/or proteoglycans containing amino acids (e.g., glycine, alanine, senrne, threonine, asparagine, glutamine, arginine, valine, isoleucine, leucine, norvaline, methionine, proline, tyrosine, hydroxyproline, and the like), collagen (e.g., gelatin), partially hydrolyzed collagen (e.g., hydrolyzed fish collagen), and collagen hydrolyzates (e.g., porcine collagen hydrolyzates).

The sweetener composition may include a surfactant additives for use in embodiments of this invention, but are not limited to, polysorbates (e.g., polyoxyethylene sorbitan monooleate (polysorbate 80), polysorbate 20, polysorbate 60), sodium dodecylbenzenesulfonate, dioctyl sulfosuccinate or dioctyl sulfosuccinate sodium, sodium dodecyl sulfate, cetylpyridinium chloride (hexadecylpyridinium chloride), hexadecyltrimethylammonium bromide, sodium cholate, carbamoyl, choline chloride, sodium glycocholate, sodium taurodeoxycholate, lauric arginate, sodium stearoyl lactylate, sodium taurocholate, lecithins, sucrose oleate esters, sucrose stearate esters, sucrose palmitate esters, sucrose laurate esters, and other emulsifiers, and the like.

A rebA formulation may include a flavonoid additives for use in embodiments of this invention generally are classified as flavonols, flavones, flavanones, flavan-3-ols, isoflavones, or anthocyanidins. Non-limiting examples of flavonoid additives include catechins (e.g., green tea extracts), polyphenols, rutins, neohesperidin, naringin, neohesperidin dihydrochalcone, and the like.

The formulation may include an alcohol additives for use in embodiments of this invention include, but are not limited to, ethanol. [00192] The formulation may include an astringent compound additives include, but are not limited to, tannic acid, europium chloride (EUC3), gadolinium chloride (GdC), terbium chloride (TbCb), alum, tannic acid, and polyphenols (e.g., tea polyphenols).

The sweetener composition may include a vitamin. Vitamins are organic compounds that the human body needs in small quantities for normal functioning. The body uses vitamins without breaking them down, unlike other nutrients such as carbohydrates and proteins. The vitamins for use in embodiment include, but not limited to, vitamin A (retinol, retinaldehyde, retinoic acid, retinoids, retinal, retinoic acid), vitamin D (vitamins D1-D5; cholecalciferol, lumisterol, ergocalciferol, dihydrotachysterol, 7-dehydrocholesterol), vitamin E (eocopherol, tocotrienol), vitamin (phylloquinone, naphthoquinone), vitamin BI (thiamin), vitamin B2 (riboflavin, vitamin G), vitamin B3 (niacin, nicotinic acid, vitamin PP), vitamin B5 (pantothenic acid), vitamin B6 (pyridoxine, pyridoxal, pyridoxamine), vitamin B7 (biotin, vitamin H), vitamin B9 (folic acid, folate, folacin, vitamin M, pteroyl-L-glutamic acid), vitamin B12 (cobalamin, cyanocobalamin), and vitamin C (ascorbic acid).

Various other compounds have been classified as vitamins by some authorities. These compounds may be termed pseudo-vitamins and include, but are not limited to, compounds such as ubiquinone (coenzyme Q10), pangamic acid, dimethylglycine, taestrile, amygdaline, flavanoids, para-aminobenzoic acid, adenine, adenylic acid, and s-methylmethionine. As used herein, the term vitamin includes pseudo-vitamins.

The formulation with rebA may include a dietary fiber. Dietary fiber, also known as bulk or roughage, is the portion of food resistant to hydrolysis by human digestive enzymes and generally comprises the indigestible portion of plant materials that moves through the digestive system and stimulates the intestine to peristalsis.

Numerous polymeric carbohydrates having significantly different structures in both composition and linkages fall within the definition of dietary fiber. Such compounds are well known to those skilled in the art, non-limiting examples of which include non-starch polysaccharides, lignin, cellulose, methylcellulose, the hemicelluloses, ?-glucans, pectins, gums, mucilage, waxes, inulin, oligosaccharides, fructooligosaccharides, cyclodextrins, chitins, and combinations thereof.

Food sources of dietary fiber include, but are not limited to, grains, legumes, fruits, and vegetables. Grains providing dietary fiber include, but are not limited to, oats, rye, barley, wheat. Legumes providing fiber include, but are not limited to, peas and beans such as soybeans. Fruits and vegetables providing a source of fiber include, but are not limited to, apples, oranges, pears, bananas, berries, tomatoes, green beans, broccoli, cauliflower, carrots, potatoes, celery. Plant foods such as bran, nuts, and seeds (such as flax seeds) are also sources of dietary fiber. Parts of plants providing dietary fiber include, but are not limited to, the stems, roots, leaves, seeds, pulp, and skin.

Although dietary fiber generally is derived from plant sources, indigestible animal products such as chitins are also classified as dietary fiber. Chitin is a polysaccharide composed of units of acetylglucosamine joined by 5(I-4) linkages, similar to the linkages of cellulose.

The sweetener composition may comprise an antioxidant. Examples of suitable antioxidants for embodiments of this invention include, but are not limited to, vitamins, vitamin cofactors, minerals, hormones, carotenoids, carotenoid terpenoids, non-carotenoid terpenoids, flavonoids, flavonoid polyphenolics (e.g., bioflavonoids), fiavonols, flavones, phenols, polyphenols, esters of phenols, esters of polyphenols, nonflavonoid phenolics, isothiocyanates, and combinations thereof. In some embodiments, the antioxidant may include vitamin A, vitamin C, vitamin E, ubiquinone, mineral selenium, manganese, melatonin, a-carotene, /̂-carotene, lycopene, lutein, zeanthin, crypoxanthin, reservatol, eugenol, quercetin, catechin, gossypol, hesperetin, curcumin, ferulic acid, thymol, hydroxytyrosol, tumeric, thyme, olive oil, lipoic acid, glutathinone, gulamine, oxalic acid, tocopherol-derived compounds, butylated hydroxyanisole, butylated hydroxyoluene, ethylenediaminetetraacetic acid, tert-butylhydroquinone, acetic acid, pectin, tocotrienol, tocopherol, coenzyme Q10, zeaxanthin, astaxanthin, canthaxantin, saponins, limonoids, kaempfedrol, myricetin, isorhamnetin, proanthocyanidins, quercetin, rutin, luteolin, apigenin, tangeritin, hesperetin, naringenin, erodictyol, flavan-3-ols (e.g., anthocyanidins), gallocatechins, epicatechin and its gallate forms, epigallocatechin and its gallate forms theaflavin and its gallate forms, thearubigins, isotlavone phytoestrogens, genistein, daidzein, glycitein, anythocyanins, cyaniding, delphinidin, malvidin, pelargonidin, peonidin, petunidin, ellagic acid, gallic acid, salicylic acid, rosmarinic acid, cinnamic acid and its derivatives (e.g., ferulic acid), chlorogenic acid, chicoric acid, gallotannins, ellagitannins, anthoxanthins, betacyanins and other plant pigments, silymarin, citric acid, lignan, antinutrients, bilirubin, uric acid, R-.alpha.-lipoic acid, N-acetylcysteine, emblicanin, apple extract, apple skin extract (applephenon), rooibos extract red, rooibos extract, green hawthorn berry extract, red raspberry extract, green coffee antioxidant, aronia extract 20% grape seed extract, cocoa extract, hops extract, mangosteen extract, mangosteen hull extract, cranberry extract, pomegranate extract, pomegranate hull extract, pomegranate seed extract, hawthorn berry extract, pomella pomegranate extract, cinnamon hark extract, grape skin extract, bilberry extract, pine bark extract, pycnogenol, elderberry extract, mulberry root extract, wolfberry (gogi) extract, blackberry extract, blueberry extract, blueberry leaf extract, raspberry extract, turmeric extract, citrus bioflavonoids, black currant, ginger, acai powder, green coffee bean extract, green tea extract, and phytic acid, or combinations thereof. In alternate embodiments, the antioxidant may comprise a synthetic antioxidant such as butylated hydroxytolune or butylated hydroxyanisole, for example. Other sources of suitable antioxidants for embodiments of this invention include, but are not limited to, fruits, vegetables, tea, cocoa, chocolate, spices, herbs, rice, organ meats from livestock, yeast, whole grains, or cereal grains.

Some antioxidants belong to the class of phytonutrients called polyphenols (also known as “polyphenolics”), which are a group of chemical substances found in plants, characterized by the presence of more than one phenol group per molecule. A variety of health benefits may derived from polyphenols, including prevention of cancer, heart disease, and chronic inflammatory disease and improved mental strength and physical strength, for example. Suitable polyphenols for embodiments of this invention, include catechins, proanthocyanidins, procyanidins, anthocyanins, quercerin, rutin, reservatrol, isoflavones, curcumin, punicalagin, ellagitannin, hesperidin, naringin, citrus flavonoids, chlorogenic acid, other similar materials, and combinations thereof.

Suitable sources of catechins for embodiments of this invention include, but are not limited to, green tea, white tea, black tea, oolong tea, chocolate, cocoa, red wine, grape seed, red grape skin, purple grape skin, red grape juice, purple grape juice, berries, pycnogenol, and red apple peel. Suitable sources of such antioxidants as proanthocyanidins and procyanidins for embodiments of this invention include, but are not limited to, red grapes, purple grapes, cocoa, chocolate, grape seeds, red wine, cacao beans, cranberry, apple peel, plum, blueberry, black currants, choke berry, green tea, sorghum, cinnamon, barley, red kidney bean, pinto bean, hops, almonds, hazelnuts, pecans, pistachio, pycnogenol, and colorful berries. Suitable sources of anthocyanins for embodiments of this invention include, but are not limited to, red berries, blueberries, bilberry, cranberry, raspberry, cherry, pomegranate, strawberry, elderberry, choke berry, red grape skin, purple grape skin, grape seed, red wine, black currant, red currant, cocoa, plum, apple peel, peach, red pear, red cabbage, red onion, red orange, and blackberries. Suitable sources of quercetin and rutin for embodiments of this invention include, but are not limited to, red apples, onions, kale, bog whortleberry, lingonberrys, chokeberry, cranberry, blackberry, blueberry, strawberry, raspberry, black currant, green tea, black tea, plum, apricot, parsley, leek, broccoli, chili pepper, berry wine, and ginkgo. Suitable sources of resveratrol for embodiments of this invention include, but are not limited to, red grapes, peanuts, cranberry, blueberry, bilberry, mulberry, Japanese Itadori tea, and red wine. Suitable sources of isoflavones for embodiments of this invention include, but are not limited to, soy beans, soy products, legumes, alfalfa spouts, chickpeas, peanuts, and red clover. Suitable sources of curcumin for embodiments of this invention include, but are not limited to, turmeric and mustard. Suitable sources of punicalagin and ellagitannin for embodiments of this invention include, but are not limited to, pomegranate, raspberry, strawberry, walnut, and oak-aged red wine. Suitable sources of citrus flavonids, such as hesperidin or naringin, for embodiments of this invention include, but are not limited to, oranges, grapefruits, and citrus juices. Suitable sources of chlorogenic acid for embodiments of this invention include, but are not limited to, green coffee, yerba mate, red wine, grape seed, red grape skin, purple grape skin, red grape juice, purple grape juice, apple juice, cranberry, pomegranate, blueberry, strawberry, sunflower, Echinacea, pycnogenol, and apple peel.

The sweetener composition may include fatty acids. As used herein, “fatty acid” refers to any straight chain monocarboxylic acid and includes saturated fatty acids, unsaturated fatty acids, long chain fatty acids, medium chain fatty acids, short chain fatty acids, fatty acid precursors (including omega-9 fatty acid precursors), and esterified fatty acids. As used herein, “long chain polyunsaturated fatty acid” refers to any polyunsaturated carboxylic acid or organic acid with a long aliphatic tail. As used herein, “omega-3 fatty acid” refers to any polyunsaturated fatty acid having a first double bond as the third carbon-carbon bond from the terminal methyl end of its carbon chain. In particular embodiments, the omega-3 fatty acid may comprise a long chain omega-3 fatty acid. As used herein, “omega-6 fatty acid” any polyunsaturated fatty acid having a first double bond as the sixth carbon-carbon bond from the terminal methyl end of its carbon chain.

The sweetener composition may include a salt. The term “salt” also refers to complexes that retain the desired chemical activity of the sweet taste improving compositions of the present invention and are safe for human or animal consumption in a generally acceptable range. Alkali metal (for example, sodium or potassium) or alkaline earth metal (for example, calcium or magnesium) salts also can be made. Salts also may include combinations of alkali and alkaline earth metals. Non-limiting examples of such salts are (a) acid addition salts formed with inorganic acids and salts formed with organic acids; (b) base addition salts formed with metal cations such as calcium, bismuth, barium, magnesium, aluminum, copper, cobalt, nickel, cadmium, sodium, potassium, and the like, or with a cation formed from ammonia, N,Nr-dibenzylethylenediamine, D-glucosamine, tetraethylammonium, or ethylenediamine; or (c) combinations of (a) and (b). Thus, any salt forms which may be derived from the sweet taste improving compositions may be used with the embodiments of the present invention as long as the salts of the sweet taste improving additives do not adversely affect the taste of the sweetener compositions comprising the at least one natural and/or synthetic high-potency sweetener. The salt forms of the additives can be added to the natural and/or synthetic sweetener composition in the same amounts as their acid or base forms.

Suitable inorganic salts may include, but are not limited to, sodium chloride, potassium chloride, sodium sulfate, potassium citrate, europium chloride (EuC), gadolinium chloride (GdCb), terbium chloride (TbCb), magnesium sulfate, alum, magnesium chloride, mono-di-, tri-basic sodium or potassium salts of phosphoric acid (e.g., inorganic phosphates), salts of hydrochloridic acid (e.g., inorganic chlorides), sodium carbonate, sodium bisulfate, and sodium bicarbonate. Furthermore, in particular embodiments, suitable organic salts useful as sweet taste improving additives include, but are not limited to, choline chloride, alginic acid sodium salt (sodium alginate), glucoheptonic acid sodium salt, gluconic acid sodium salt (sodium gluconate), gluconic acid potassium salt (potassium gluconate), guanidine HC1, glucosamine HC1, amriloride HC1, monosodium glutamate, adenosine monophosphate salt, magnesium gluconate, potassium tartrate (monohydrate), and sodium tartrate (dihydrate).

The sweetener composition can be applied as high intensity sweetener to produce zero calorie, reduced calorie or diabetic beverages and food products with improved taste characteristics. Also it can be used in drinks, foodstuffs, pharmaceuticals, and other products in which sugar cannot be used.

The sweetener composition can be used as a sweetener not only for drinks, foodstuffs, and other products dedicated for human consumption, but also in animal feed and fodder with improved characteristics.

The sweetener composition can be used as sweetening compound can be as alcoholic beverages such as vodka, wine, beer, liquor, sake, etc; natural juices, refreshing drinks, carbonated soft drinks, diet drinks, zero calorie drinks, reduced calorie drinks and foods, yogurt drinks, instant juices, instant coffee, powdered types of instant beverages, canned products, syrups, fermented soybean paste, soy sauce, vinegar, dressings, mayonnaise, ketchups, curry, soup, instant bouillon, powdered soy sauce, powdered vinegar, types of biscuits, rice biscuit, crackers, bread, chocolates, caramel, candy, chewing gum, jelly, pudding, preserved fruits and vegetables, fresh cream, jam, marmalade, flower paste, powdered milk, ice cream, sorbet, vegetables and fruits packed in bottles, canned and boiled beans, meat and foods boiled in sweetened sauce, agricultural vegetable food products, seafood, ham, sausage, fish ham, fish sausage, fish paste, deep fried fish products, dried seafood products, frozen food products, preserved seaweed, preserved meat, tobacco, medicinal products, and many others. In principal it can have unlimited applications.

The sweetened composition comprises a beverage, non-limiting examples of which include non-carbonated and carbonated beverages such as colas, ginger ales, root beers, ciders, fruit-flavored soft drinks (e.g., citrus-flavored soft drinks such as lemon-lime or orange), powdered soft drinks, and the like; fruit juices originating in fruits or vegetables, fruit juices including squeezed juices or the like, fruit juices containing fruit particles, fruit beverages, fruit juice beverages, beverages containing fruit juices, beverages with fruit flavorings, vegetable juices, juices containing vegetables, and mixed juices containing fruits and vegetables; sport drinks, energy drinks, near water and the like drinks (e.g., water with natural or synthetic flavorants); tea type or favorite type beverages such as coffee, cocoa, black tea, green tea, oolong tea and the like;

beverages containing milk components such as milk beverages, coffee containing milk components, cafe au lait, milk tea, fruit milk beverages, drinkable yogurt, lactic acid bacteria beverages or the like; and dairy products.

Generally, the amount of sweetener composition present in a sweetened composition varies widely depending on the particular type of sweetened composition and its desired sweetness. Those of ordinary skill in the art can readily discern the appropriate amount of sweetener to put in the sweetened composition.

The sweetener composition can be used in dry or liquid forms. It can be added before or after heat treatment of food products. The amount of the sweetener depends on the purpose of usage. It can be added alone or in the combination with other compounds.

During the manufacturing of foodstuffs, drinks, pharmaceuticals, cosmetics, table top products, chewing gum the conventional methods such as mixing, kneading, dissolution, pickling, permeation, percolation, sprinkling, atomizing, infusing and other methods can be used.

Thus, products of the present invention can be made by any method known to those skilled in the art that provide homogenous even or homogeneous mixtures of the ingredients. These methods include dry blending, spray drying, agglomeration, wet granulation, compaction, co-crystallization and the like.

In solid form the sweetening composition of the present invention can be provided to consumers in any form suitable for delivery into the comestible to be sweetened, including sachets, packets, bulk bags or boxes, cubes, tablets, mists, or dissolvable strips. The composition can be delivered as a unit dose or in bulk form.

For liquid sweetener systems and compositions convenient ranges of fluid, semi-fluid, paste and cream forms, appropriate packing using appropriate packing material in any shape or form shall be invented which is convenient to carry or dispense or store or transport any combination containing any of the above sweetener products or combination of product produced above.

The sweetener composition may include various bulking agents, functional ingredients, colorants, flavors. A reference herein to a patent document or other matter which is given as prior art is not to be taken as an admission that that document or matter was known or that the information it contains was part of the common general knowledge as at the priority date of any of the claims.

The disclosure of each reference set forth herein is incorporated herein by reference in its entirety.

The following Examples illustrate preferred embodiments of the invention for the fermentatively produced and purified Rebaudioside A as per product specifications and related compounds and the use thereof in foodstuffs and pharmaceuticals. Accordingly, the present invention is further illustrated by the following Examples:

### EXAMPLES

### General

Standard genetic techniques, such as overexpression of enzymes in the host cells, as well as for additional genetic modification of host cells, are known methods in the art, such as described in Sambrook and Russel (2001) “Molecular Cloning: A Laboratory Manual (3^{rd} edition), Cold Spring Harbor Laboratory, Cold Spring Harbor Laboratory Press, or F. Ausubel et al, eds., “Current protocols in molecular biology”, Green Publishing and Wiley Interscience, New York (1987). Methods for transformation and genetic modification of fungal host cells are known from e.g. EP-A-0 635 574, WO 98/46772, WO 99/60102 and WO 00/37671.

A description of the sequences is set out in Table 1. Sequences described herein may be defined with reference to the sequence listing or with reference to the database accession numbers also set out in Table 1.

### Example 1

### Over-Expression of ERG20, BTS1 and tHMG in S. cerevisiae

For over-expression of ERG20, BTS1 tHMG1, expression cassettes were designed to be integrated in one locus using technology described in co-pending patent application no. PCT/EP2013/056623. To amplify the 5′ and 3′ integration flanks for the integration locus, suitable primers and genomic DNA from a CEN.PK yeast strain (van Dijken et al. Enzyme and Microbial Technology 26 (2000) 706-714) was used. The different genes were ordered as cassettes (containing homologous sequence, promoter, gene, terminator, homologous sequence) at DNA2.0. The genes in these cassettes were flanked by constitutive promoters and terminators. See Table 2. Plasmid DNA from DNA2.0 containing the ERG20, tHMG1 and BTS1 cassettes were dissolved to a concentration of 100 ng/μl. In a 50 μl PCR mix 20 ng template was used together with 20 pmol of the primers. The material was dissolved to a concentration of 0.5 μg/μl.

TABLE 2
Composition of the over-expression constructs.

| Promoter | ORF | Terminator |
|---|---|---|
| Eno2 | Erg20 | Adh1 |
| (SEQ ID NO: 201) | (SEQ ID NO: 81) | (SEQ ID NO: 212) |
| Fba1 | tHMG1 | Adh2 |
| (SEQ ID NO: 202) | (SEQ ID NO: 79) | (SEQ ID NO: 213) |
| Tef1 | Bts1 | Gmp1 |
| (SEQ ID NO: 203) | (SEQ ID NO: 83) | (SEQ ID NO: 214) |

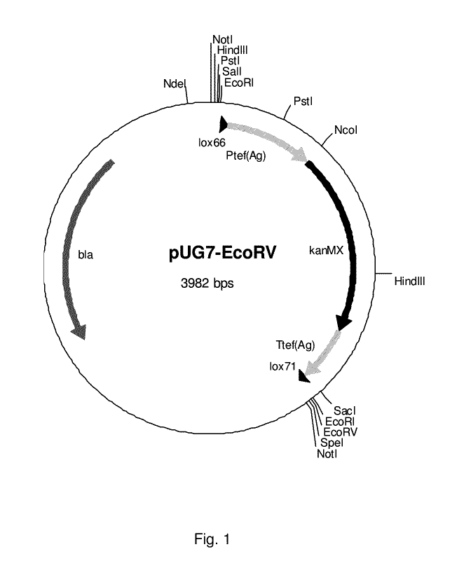

For amplification of the selection marker, the pUG7-EcoRV construct (FIG. 1) and suitable primers were used. The KanMX fragment was purified from gel using the Zymoclean Gel DNA Recovery kit (ZymoResearch). Yeast strain Cen.PK113-3C was transformed with the fragments listed in Table 3.

TABLE 3
DNA fragments used for transformation
of ERG20, tHMG1 and BTS1

Fragment
5′YPRcTau3
ERG20 cassette
tHMG1 cassette
KanMX cassatte
BTS1 cassette
3′YPRcTau3

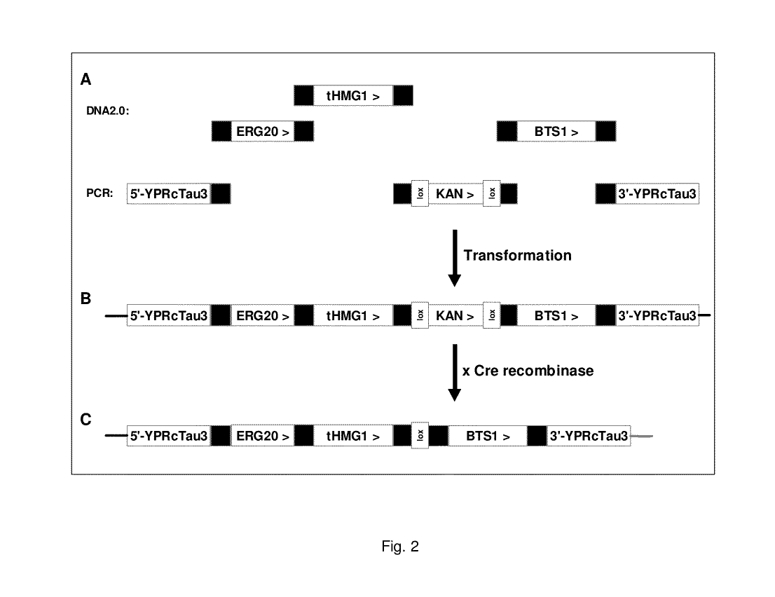

After transformation and recovery for 2.5 hours in YEPhD (yeast extract phytone peptone glucose; BBL Phytone Peptone from BD) at 30° C. the cells were plated on YEPhD agar with 200 μg/ml G418 (Sigma). The plates were incubated at 30° C. for 4 days. Correct integration was established with diagnostic PCR and sequencing. Over-expression was confirmed with LC/MS on the proteins. The schematic of the assembly of ERG20, tHMG1 and BTS1 is illustrated in FIG. 2. This strain is named STV002.

Expression of the CRE-recombinase in this strain led to out-recombination of the KanMX marker. Correct out-recombination, and presence of ERG20, tHMG and BTS1 was established with diagnostic PCR.

### Example 2

### Knock Down of Erg9

For reducing the expression of Erg9, an Erg9 knock down construct was designed and used that contains a modified 3′ end, that continues into the TRP1 promoter driving TRP1 expression.

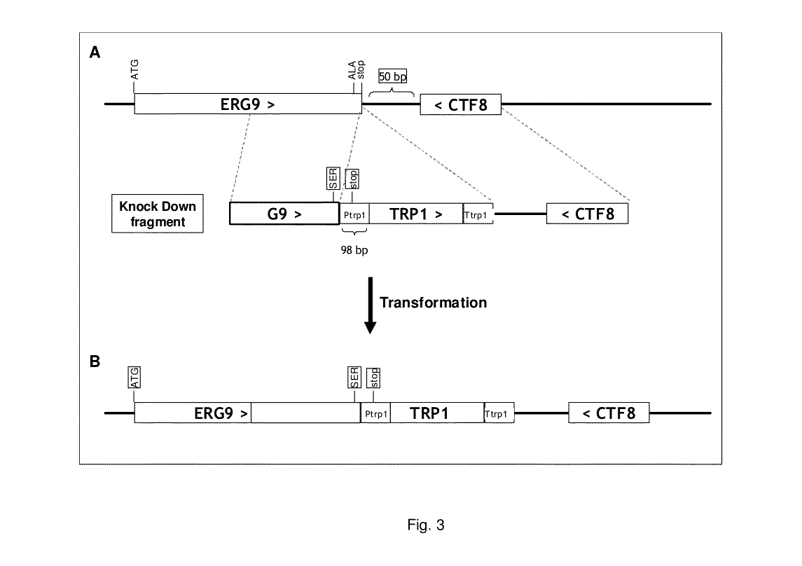

The construct containing the Erg9-KD fragment was transformed to E. coli TOP10 cells. Transformants were grown in 2PY (2 times Phytone peptone Yeast extract), sAMP medium. Plasmid DNA was isolated with the QIAprep Spin Miniprep kit (Qiagen) and digested with SalI-HF (New England Biolabs). To concentrate, the DNA was precipitated with ethanol. The fragment was transformed to S. cerevisiae, and colonies were plated on mineral medium (Verduyn et al, 1992. Yeast 8:501-517) agar plates without tryptophan. Correct integration of the Erg9-KD construct was confirmed with diagnostic PCR and sequencing. The schematic of performed transformation of the Erg9-KD construct is illustrated in FIG. 3. The strain was named STV003.

### Example 3

### Over-Expression of UGT2_1a

For over-expression of UGT2_1a, technology was used as described in co-pending patent application nos. PCT/EP2013/056623 and PCT/EP2013/055047. The UGT2_1a was ordered as a cassette (containing homologous sequence, promoter, gene, terminator, homologous sequence) at DNA2.0. For details, see Table 4. To obtain the fragments containing the marker and Cre-recombinase, technology was used as described in co-pending patent application no. PCT/EP2013/055047. The NAT marker, conferring resistance to nourseothricin was used for selection.

TABLE 4
Composition of the over-expression construct

| Promoter | ORF | Terminator |
|---|---|---|
| Pgk1 | UGT2_1a | Adh2 |
| (SEQ ID NO: 204) | (SEQ ID NO: 87) | (SEQ ID NO: 213) |

Suitable primers were used for amplification. To amplify the 5′ and 3′ integration flanks for the integration locus, suitable primers and genomic DNA from a CEN.PK yeast strain was used.

S. cerevisiae yeast strain STV003 was transformed with the fragments listed in Table 5, and the transformation mix was plated on YEPhD agar plates containing 50 μg/ml nourseothricin (Lexy NTC from Jena Bioscience).

TABLE 5
DNA fragments used for transformation of UGT2_1a

Fragment
5′Chr09.01
UGT2_1a cassette
NAT-CR
RE
3′Chr09.01

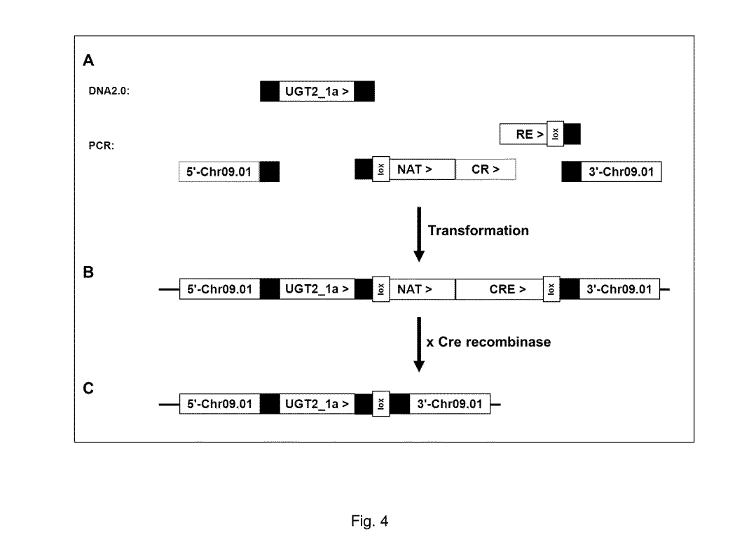

Expression of the CRE recombinase is activated by the presence of galactose. To induce the expression of the CRE recombinase, transformants were restreaked on YEPh Galactose medium. This resulted in out-recombination of the marker(s) located between lox sites. Correct integration of the UGT2a and out-recombination of the NAT marker was confirmed with diagnostic PCR. The resulting strain was named STV004. The schematic of the performed transformation of the UGT2_1a construct is illustrated in FIG. 4.

### Example 4

### Over-Expression of Production Pathway to RebA: CPS, KS, KO, KAH, CPR, UGT1, UGT3 and UGT4

All pathway genes leading to the production of RebA were designed to be integrated in one locus using technology described in co-pending patent application no. PCT/EP2013/056623. To amplify the 5′ and 3′ integration flanks for the integration locus, suitable primers and genomic DNA from a CEN.PK yeast strain was used. The different genes were ordered as cassettes (containing homologous sequence, promoter, gene, terminator, homologous sequence) at DNA2.0 (see Table 5 for overview). The DNA from DNA2.0 was dissolved to 100 ng/μl. This stock solution was further diluted to 5 ng/μl, of which 1 μl was used in a 50 μl-PCR mixture. The reaction contained 25 pmol of each primer. After amplification, DNA was purified with the NucleoSpin 96 PCR Clean-up kit (Macherey-Nagel) or alternatively concentrated using ethanol precipitation.

TABLE 6
Sequences used for production pathway to RebA

| | | SEQ | |
|---|---|---|---|
| Promoter | ORF | ID | Terminator |
| Kl prom 12.pro | trCPS_SR | 61 | Sc ADH2.ter |
| (SEQ ID NO: 205) | | | (SEQ ID NO:) |
| Sc PGK1.pro | trKS_SR | 65 | Sc TAL1.ter |
| (SEQ ID NO: 204) | | | (SEQ ID NO: 215) |
| Sc ENO2.pro | KO_2 | 23 | Sc TPI1.ter |
| (SEQ ID NO: 201) | | | (SEQ ID NO: 216) |
| Ag lox_TEF1.pro | KANMX | 211 | Ag TEF1_lox.ter |
| (SEQ ID NO: 206) | | | (SEQ ID NO: 217) |
| Sc TEF1.pro | KAH_4 | 33 | Sc GPM1.ter |
| (SEQ ID NO: 203) | | | (SEQ ID NO: 214) |
| Kl prom 6.pro | CPR_SR | 59 | Sc PDC1.ter |
| (SEQ ID NO: 207) | | | (SEQ ID NO: 218) |
| Kl prom 3.pro | UGT1_SR | 71 | Sc TDH1.ter |
| (SEQ ID NO: 221) | | | (SEQ ID NO: 219) |
| Kl prom 2.pro | UGT3_SR | 73 | Sc ADH1.ter |
| (SEQ ID NO: 222) | | | (SEQ ID NO: 212) |
| Sc FBA1.pro | UGT4_SR | 75 | Sc ENO1.ter |
| (SEQ ID NO: 202) | | | (SEQ ID NO: 220) |

All fragments for the pathway to RebA, the marker and the flanks (see overview in Table 7) were transformed to S. cerevisiae yeast strain STV004. After overnight recovery in YEPhD at 20° C. the transformation mixes were plated on YEPhD agar containing 200 μg/ml G418. These were incubated 3 days at 25° C. and one night at RT.

TABLE 7
DNA fragments used for transformation of CPS,
KS, KO, KanMX, KAH, CPR, UGT1, UGT3 and UGT4.

Fragment
5′INT1
CPS cassette
KS cassette
KO cassette
KanMX cassette
KAH cassette
CPR cassette
UGT1 cassette
UGT3 cassette
UGT4 cassette
3′INT1

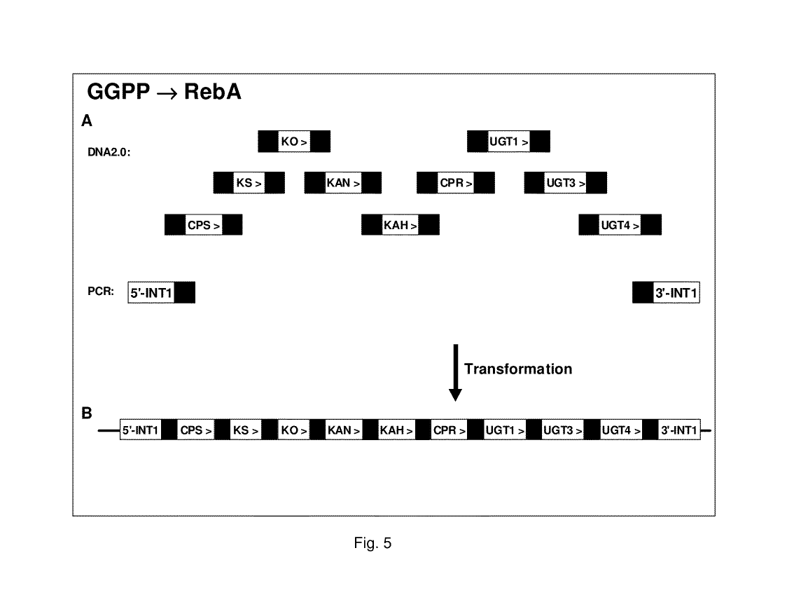

Correct integration was confirmed with diagnostic PCR and sequence analysis (3500 Genetic Analyzer, Applied Biosystems). The sequence reactions were done with the BigDye Terminator v3.1 Cycle Sequencing kit (Life Technologies). Each reaction (10 μl) contained 50 ng template and 3.2 pmol primer. The products were purified by ethanol/EDTA precipitation, dissolved in 10 μl HiDi formamide and applied onto the apparatus. The strain was named STV016. The schematic of how the pathway from GGPP to RebA is integrated into the genome is illustrated in FIG. 5.

### Example 5

### Construction of Strain STV027

To remove the KanMX marker from the chromosome of strain STV016, this strain was transformed with plasmid pSH65, expressing Cre-recombinase (Güldender, 2002). Subsequently plasmid pSH65 was cured from the strain by growing on non-selective medium (YEP 2% glucose). The resulting, KanMX-free and pSH65-free strains, as determined by plating on plates containing 200 μg G418/ml or 20 μg phleomycin/ml, where no growth should occur, was named STV027. Absence of the KanMX marker was furthermore confirmed with diagnostic PCR.

### Example 6

### Preparation of Fermentative Steviol Glycoside Compositions

The microbial production strain STV027 constructed as described above is used for production of fermentative RebaudiosideA. The pH is controlled at 5.0 by addition of ammonia (12.5 wt %). Temperature is controlled at 27° C. pO2 is controlled at 40% by adjusting the stirrer speed. Glucose concentration is kept limited by controlled feed to the fermenter. Subsequently, 6 ml of the content of the shake-flask is transferred into a fermenter (starting volume 0.3 L), which contained the medium as set out in the Examples of PCT/EP2013/051262. The pH is controlled between pH 4.0 and pH 8.0 by addition of ammonia (12.5 wt %). Temperature is controlled between 20 and 45° C. p02 is controlled between 05-40% by adjusting the stirrer speed. Glucose concentration is kept limited by controlled feed to the fermenter. After the completion of fermentation, microbial production host cells are removed and the fermentation broth was processed as per the unit operations illustrated in FIG. 7 and/or modification thereof. In the case of Example 7, microbial production host cells were removed and the fermentation broth was processed as per Example 7 of U.S. Ser. No. 13/956,144.

Fermentatively produced Reb-A was analytically characterized meeting purity and product specification using methods known in the art.

### Example 7

### Comparison of Fermentatively-Produced Rebaudioside A with Plant Derived Rebaudioside A in Four Applications

Fermentatively-produced rebA was used as described in Examples 1 to 6. The plant derived product used was Reb A 97% from Pure Circle/Prinova.

### 7.1 Products Tested

### Acidified Water

| Acidified water | g/l |
|---|---|
| Citric Acid | 0.6 |
| Reb A | 0.24 |

### Procedure for Preparation of the Beverage:

One liter of water was weighed out and citric acid and rebaudioside A dissolved while stirring with a standard mixer. No further processing was needed.

### Near Water

| Mixed Berry Sobe Knockoff | g/l |
|---|---|
| Filtered Water | 984.5 |
| Erythritol | 13 |
| Citric Acid | 1 |
| Mixed Berry | 1 |
| Canthaxanthin 10% CWS/S | 1.5 |
| Stock Solution | |
| Xanthan Gum | 0.4 |
| Reb A | 0.3 |
| Stevia Masker Flavor | 1 |

### Procedure for Preparation of the Beverage:

60% of the required water was weighed out. High shear xanthan gum was then added into the water (30 seconds at 600 rpm)
While mixing with a standard mixer, eythritol, citric acid, flavor, Canthaxanthin stock solution (see for preparation below), stevia and the stevia masking flavor were added
the remaining water was then added and the resulting composition was mixed well
the resulting composition was pasteurized at 95° C. for 30 seconds and hot filled into bottles.

### Procedure for Preparation of Stock Solution:

Weigh out specified product form (Canthaxanthin 10%-CWS/S)
In a beaker, measure out 60 ml of warm deionized water 45° C. to 55° C.
Slowly add the powder to the warm water while stirring. Minimize incorporation of air while stirring
Stir for 10-15 minutes to ensure complete dispersion of the powder
Fill to 100 ml total volume with room temperature water and then stir.

### Juice

| 50 cal bev Pomegranate (45% Juice) | g/l |
|---|---|
| Filtered Water | 922.02 |
| Apple juice concentrate | 34.29 |
| Pomegranate Juice Concentrate | 32.1 |
| Grape Juice Concentrate | 32.1 |
| Pomegranate Flavor | 1 |
| Malic Acid | 0.5 |
| Citric Acid | 0.5 |
| Reb A | 0.2 |
| Stevia Masker Flavor | 1 |

### Procedure for Preparation of Beverage:

water was weighed out and then stevia, stevia masking flavor, pomegranate flavor, citric acid and malic acid was added
apple juice concentrate, pomegranate juice concentrate and grape juice concentrate was then added while using a standard mixer
the resulting composition was mixed well, pasteurized at 95° C. for 30 seconds and then hot filled into bottles

### Cola

| Reduced Calorie Cola | g/l |
|---|---|
| Filtered Water | 944.31 |
| Car Color DS400 | 0.8 |
| Sugar | 78.75 |
| Phosphoric Acid (85%) | 1.1 |
| Reb A 97% | 0.135 |
| Nat. Anhydrous Caffeine | 0.095 |
| (57 mg per 20 oz) | |
| Tri-Sodium Citrate | 0.46 |
| Potassium Sorbate | 0.25 |
| Cola Flavor | 2.5 |
| Stevia Masker Flavor | 1 |

### Procedure for Preparation of Beverage:

the water was weighed out and then potassium sorbate was dissolved in the water while stirring. Next stevia, tri-sodium citrate and caffeine were dissolved
sugar, caramel color, stevia masking flavor, cola flavor and phosphoric acid were then added
the resulting composition was then mixed well, carbonated to 3.6 Carbonation Units and then filled into plastic bottles.

### 7.2 Sensory Evaluation Method

Six experienced and trained panelists evaluated all samples with the Sensory Spectrum® method which is used for detailed flavor analysis. Per application type, each sample was evaluated twice by each panelist in two different sessions. Scoring was done on a 0 (very low intensity) to 15 (very high intensity) Sensory Spectrum scale with discussions to reach consensus on the scores. The products were presented in a balanced order.

Sweetness was rated after the products was held in the mouth for 3 seconds after which the product was expectorated. The sweetness score, also on a scale ranging from 0 to 15, is the mean intensity score of individual data. To evaluate the significance of the difference an ANOVA was done.

### 7.3 Results

### Overall Sweetness

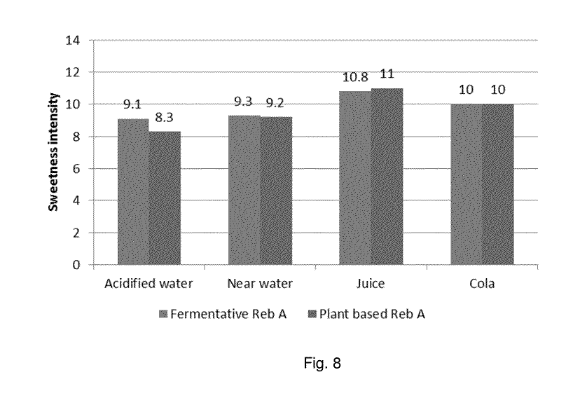

In FIG. 8, the sweetness per sample is shown. No significant differences on sweetness (p<0.05) were found between the fermentative and plant based Reb A in each of the tested applications.

### Results Per Application

### Acidified Water

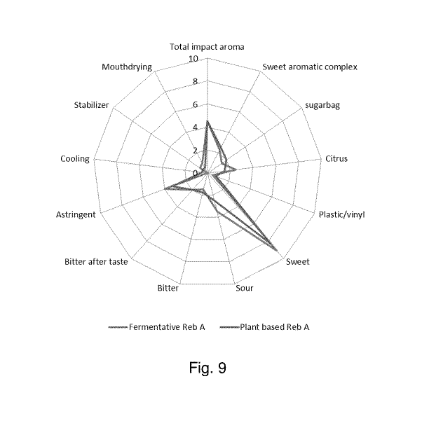

In FIG. 9, the consensus data for the acidified water application is shown. Fermentative Reb A showed an impact on citrus and sour attributes, indicating an enhancement effect of fermentative Reb A based on the used ingredients.

### Near Water

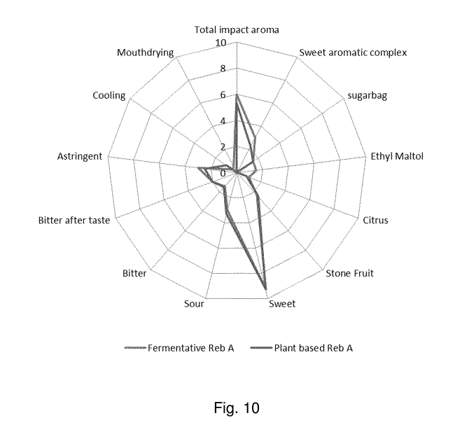

In FIG. 10, the consensus data for the near water application is shown. Fermentative Reb A showed an impact on total aroma impact, sweet aromatic complex and ethyl maltol (also known as strawberry flavor), indicating an enhancement effect of fermentative Reb A based on the used ingredients.

### Juice

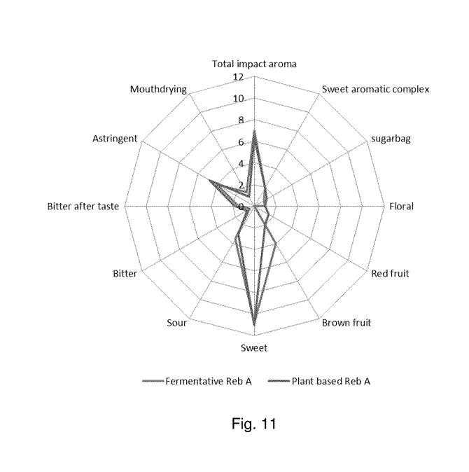

In FIG. 11, the consensus data for the juice application is shown. Fermentative Reb A showed an impact on total aroma impact and brown fruit, indicating an enhancement effect of fermentative Reb A based on the used ingredients.

The raw data is shown in Tables 8, 9 and 10 below

TABLE 8
Raw data for acidified water

| Acidified water | Fermentative Reb A | Plant based Reb A |
|---|---|---|
| Total impact aroma | 4.5 | 4.5 |
| Sweet aromatic complex | 2.3 | 2.5 |
| sugarbag | 1.5 | 2 |
| Citrus | 2.5 | 1.5 |
| Plastic/vinyl | 0.5 | 0.8 |
| Sweet | 9.1 | 8.3 |
| Sour | 3.5 | 2.5 |
| Bitter | 1.5 | 1.8 |
| Bitter after taste | 2 | 2 |
| Astringent | 4 | 3.3 |
| Cooling | 0 | 0.5 |
| Stabilizer | 0.8 | 0.5 |
| Mouthdrying | 1 | 0.5 |

TABLE 9
Raw data for acidified water

| Near water | Fermentative Reb A | Plant based Reb A |
|---|---|---|
| Total impact aroma | 6 | 5.3 |
| Sweet aromatic complex | 3 | 2.3 |
| sugarbag | 1.5 | 1.5 |
| Ethyl Maltol | 1.5 | 0 |
| Citrus | 1 | 0.8 |
| Stone Fruit | 2.3 | 2.5 |
| Sweet | 9.3 | 9.2 |
| Sour | 3 | 3.3 |
| Bitter | 1.4 | 1.5 |
| Bitter after taste | 2 | 2 |
| Astringent | 3 | 2.5 |
| Cooling | 0.4 | 1 |
| Mouthdrying | 0.5 | 0 |

TABLE 10
Raw data for acidified water

| Juice | Fermentative Reb A | Plant based Reb A |
|---|---|---|
| Total impact aroma | 7 | 6.3 |
| Sweet aromatic complex | 2 | 2 |
| sugarbag | 1 | 1.3 |
| Floral | 1 | 1 |
| Red fruit | 0 | 1.5 |
| Brown fruit | 4 | 2 |
| Sweet | 10.8 | 11 |
| Sour | 3.5 | 3 |
| Bitter | 0.5 | 0.8 |
| Bitter after taste | 1.5 | 1.8 |
| Astringent | 4 | 4.8 |
| Mouthdrying | 1.5 | 1 |

### Example 8

### Low-Calorie Orange Juice Drink

60 g of concentrated orange juice is mixed with 1.1 g of citric acid, 0.24 g of vitamin C, 1.0 g of orange essence, 0.76 g of fermentatively-produced Rebaudioside A and water, to create a homogeneously dissolved mixture of 1000 mL total amount. Then, the mixture is pasteurized for a period of 20 seconds at about 95.degree centigrade in order to prepare an orange juice similar to one made by conventional method. The product is subjected to sensory evaluation in terms of flavour, aftertaste and mouthfeel. The data shows that excellent taste and mouth-feel results were obtained for fermentatively produced Rebaudioside A.

Juices from other fruits, such as apple, lemon, apricot, cherry, pineapple, etc. can be prepared using the same approach.

### Example 8

### Ice-Cream

1.50 kg of whole milk is heated to 45° C., and 300 grams of milk cream, 100 grams of tagatose, 90 grams of sorbitol, 6 grams of carrageenan as a stabilizer, 3 grams of polysorbate-80 as an emulsifier and 1.0 gram of fermentatively-produced Rebaudioside A are added into the milk and stirred until the ingredients completely dissolved.

The mixture is then pasteurized at a temperature of 80° C. for 25 seconds. After homogenization the samples are kept at a temperature of 4° C. for 24 hours to complete the aging process. Vanilla flavor (1.0% of the mixture weight) and coloring (0.025% of the mixture weight) are added into the mixture after aging. The mixture is then transferred to an icecream maker to produce icecream automatically. Samples of produced ice creams are transferred to sealed containers and are kept in the freezer at a temperature of −18° C.

The physicochemical properties of the ice cream, as well as the overall attributes of color, smoothness, surface texture, air cell, vanilla aroma intensity, vanilla taste, chalkiness, iciness and melting rate are assessed.

### Example 9

### Yoghurt

In 1 kg of defatted milk, 0.8 grams of fermentatively-produced Rebaudioside A, prepared according to the invention is dissolved. After pasteurization at 82° C. for 20 minutes, the milk was cooled to 40° C. A starter in an amount of 30 grams is added and the mixture is incubated at 37° C. for 6 hours. Then, the fermented mass is maintained at 10-15° C. for 12 hours.

The product is a low-calorie and low-cariogenic yoghurt and is assessed for taste and odor.

### Example 10

### Ice Lemon Tea

The formula for the beverage is as below:
95% high purity fermentatively-produced Rebaudioside A, 0.08 Sodium benzoate, 0.02 Citric acid, 0.27 Ascorbic acid, 0.01 Tea extract, 0.03 Lemon flavor 0.10 Water to 100

All ingredients are blended and dissolved in the water and pasteurized. The product is assessed for taste and flavour. Sensory and physicochemical characteristics are compared to that of caloric lemon-flavored iced tea.

### Example 11

### Bread

1 kg of flour, 37.38 grams of fructooligosaccharide syrup, 80 grams of margarine, 20 grams of salt, 20 grams of yeasts, and 0.25 grams of 95% high purity fermentatively produced Rebaudioside A, obtained as described above are placed into a blender and mixed well. 600 ml of water is poured into the mixture and kneaded sufficiently. At the completion of the kneading process, the dough is shaped and raised for 30 to 45 minutes. The ready dough is placed in an oven and baked for 45 minutes. Bread samples are assessed for color and texture.

### Example 12

### Diet to Mid Caloric Cookie

Flour, 50.0%; margarine, 30.0%; fructose, 10.0%; maltitol, 8.0%; whole milk, 1.0%; salt, 0.2%; baking powder, 0.15%; vanillin, 0.1%; fermentatively-produced Rebaudioside A, 0.55% obtained according to this invention are kneaded well in a dough-mixing machine. After moulding of the dough, the cookies are baked at 200° C. for 15 minutes.

The product is a low-calorie diet cookie and is assessed for taste and appropriate sweetness.

### Example 13

### Soy Sauce

0.8 g of fermentatively-produced Rebaudioside A is added to 1000 mL of soy sauce and mixed homogenously. The product is assessed for taste and texture.

### Example 14

### Chocolate

A composition containing 30 kg of cacao liquor, 11.5 kg of cacao butter, 14 kg of milk powder, 44 kg of sorbitol, 0.1 kg of salt, and 0.1 kg of fermentatively produced Rebaudioside-A is kneaded sufficiently and the mixture is then placed in a refiner to reduce its particle size for 24 hours. Thereafter, the content is transferred into a conche 300 grams of lecithin is added, and the composition is kneaded at 50° C. for 48 hours. Then, the content is placed in a shaping apparatus and solidified.

The products are low-cariogenic and low-calorie chocolate and are assessed for texture and the presence of any after-taste.

### Example 15

### Tooth Paste

A tooth paste is prepared by kneading a composition comprising of calcium phosphate, 45.0%; carboxymethylcellulose, 1.5%; carrageenan, 0.5%; glycerol, 18.0%; polyoxyethylene sorbitan mono-ester, 2.0%; beta-cyclodextrin, 1.5%; sodium laurylsarcosinate, 0.2%; flavoring, 1.0%; preservative, 0.1%; fermentatively-produced Rebaudioside A, obtained according to this invention, 0.2%; and water to 100%, by usual way.

The product is assessed for foaming and cleaning abilities along with appropriate sweetness.

### Example 16

### Low-Calorie Carbonated Drink

The formula for the beverage was as below:

| Ingredients | Quantity, % |
|---|---|
| Cola flavor | 0.340 |
| Phosphoric acid (85%) | 0.100 |
| Sodium citrate | 0.310 |
| Sodium benzoate | 0.018 |
| Citric acid | 0.018 |
| Sweetener | 0.030 |
| Carbonated water | to 100 |

The beverages are prepared with different sweeteners (plant-extracted Rebaudioside A (95%) and fermentatively-produced Rebaudioside A (95%)) and given to an 8 judge panel for comparison. The beverages are assessed for bitter taste, astringent taste, after-taste, quality of the sweet taste and the overall evaluation,

The above assessments set out in Examples 7 to 17 are to demonstrate that the various products prepared using fermentatively-produced Rebaudioside A possess improved organoleptic characteristics as compared with similar products made with plant-extracted similar grade Rebaudioside A.

TABLE 1
Description of the sequence listing

| Nucleic acid | Nucleic | | | | |
|---|---|---|---|---|---|
| (CpO for S. | acid (CpO for | Amino | | | |
| cerevisiae) | Y. lipolytica) | acid | Id* | UniProt\hat{ } | Organism |
| SEQ ID NO: | SEQ ID NO: | SEQ ID | CPS_1 | Q9FXV9 | Lactuca sativa |
| 1 | 151 | NO: 2 | | | (Garden Lettuce) |
| SEQ ID NO: 3 | SEQ ID NO: 152 | SEQ ID NO: 4 | tCPS_1 | 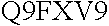 | Lactuca sativa (Garden Lettuce) |
| SEQ ID NO: | SEQ ID NO: | SEQ ID | CPS_2 | D2X8G0 | Picea glauca |
| 5 | 153 | NO: 6 | | | |
| SEQ ID NO: | SEQ ID NO: | SEQ ID | CPS_3 | Q45221 | Bradyrhizobium |
| 7 | 154 | NO: 8 | | | japonicum |
| SEQ ID NO: | SEQ ID NO: | SEQ ID | KS_1 | Q9FXV8 | Lactuca sativa |
| 9 | 155 | NO: 10 | | | (Garden Lettuce) |
| SEQ ID NO: 11 | SEQ ID NO: 156 | SEQ ID NO: 12 | tKS_1 | 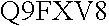 | Lactuca sativa (Garden Lettuce) |
| SEQ ID NO: | SEQ ID NO: | SEQ ID | KS_2 | D2X8G1 | Picea glauca |
| 13 | 157 | NO: 14 | | | |
| SEQ ID NO: | SEQ ID NO: | SEQ ID | KS_3 | Q45222 | Bradyrhizobium |
| 15 | 158 | NO: 16 | | | japonicum |
| SEQ ID NO: | SEQ ID NO: | SEQ ID | CPSKS_1 | O13284 | Phaeosphaeria sp |
| 17 | 159 | NO: 18 | | | |
| SEQ ID NO: | SEQ ID NO: | SEQ ID | CPSKS_2 | Q9UVY5 | Gibberella fujikuroi |
| 19 | 160 | NO: 20 | | | |
| SEQ ID NO: | SEQ ID NO: | SEQ ID | KO_1 | B5MEX5 | Lactuca sativa |
| 21 | 161 | NO: 22 | | | (Garden Lettuce) |
| SEQ ID NO: | SEQ ID NO: | SEQ ID | KO_2 | B5MEX6 | Lactuca sativa |
| 23 | 162 | NO: 24 | | | (Garden Lettuce) |
| SEQ ID NO: | SEQ ID NO: | SEQ ID | KO_3 | B5DBY4 | Sphaceloma |
| 25 | 163 | NO: 26 | | | manihoticola |
| SEQ ID NO: | SEQ ID NO: | SEQ ID | KAH_1 | Q2HYU7 | Artemisia annua |
| 27 | 164 | NO: 28 | | | (Sweet wormwood). |
| SEQ ID NO: | SEQ ID NO: | SEQ ID | KAH_2 | B9SBP0 | Ricinus communis |
| 29 | 165 | NO: 30 | | | (Castor bean). |
| SEQ ID NO: | SEQ ID NO: | SEQ ID | KAH_3 | Q0NZP1 | Stevia rebaudiana |
| 31 | 166 | NO: 32 | | | |
| SEQ ID NO: | SEQ ID NO: | SEQ ID | KAH_4 | JP2009065886 | Arabidopsis thaliana |
| 33 | 167 | NO: 34 | | | (Mouse-ear cress) |
| SEQ ID NO: | SEQ ID NO: | SEQ ID | UGT1_1 | A9X3L6 | Ixeris dentata var. |
| 35 | 168 | NO: 36 | | | albiflora. |
| SEQ ID NO: | SEQ ID NO: | SEQ ID | UGT1_2 | B9SIN2 | Ricinus communis |
| 37 | 169 | NO: 38 | | | (Castor bean). |
| SEQ ID NO: | SEQ ID NO: | SEQ ID | UGT3_1 | A9X3L7 | Ixeris dentata var. |
| 39 | 170 | NO: 40 | | | Albiflora |
| SEQ ID NO: | SEQ ID NO: | SEQ ID | UGT3_2 | B9IEM5 | Populus trichocarpa |
| 41 | 171 | NO: 42 | | | (Western balsam poplar) |
| SEQ ID NO: | SEQ ID NO: | SEQ ID | UGT3_3 | Q9M6E7 | Nicotiana tabacum |
| 43 | 172 | NO: 44 | | | |
| SEQ ID NO: | SEQ ID NO: | SEQ ID | UGT3_4 | A3E7Y9 | Vaccaria hispanica |
| 45 | 173 | NO: 46 | | | |
| SEQ ID NO: | SEQ ID NO: | SEQ ID | UGT3_5 | P10249 | Streptococcus mutans |
| 47 | 174 | NO: 48 | | | |
| SEQ ID NO: | SEQ ID NO: | SEQ ID | UGT4_1 | A4F1T4 | Lobelia erinus |
| 49 | 175 | NO: 50 | | | (Edging lobelia) |
| SEQ ID NO: | SEQ ID NO: | SEQ ID | UGT4_2 | Q9M052 | Arabidopsis thaliana |
| 51 | 176 | NO: 52 | | | (Mouse-ear cress) |
| SEQ ID NO: | SEQ ID NO: | SEQ ID | CPR_1 | Q7Z8R1 | Gibberella fujikuroi |
| 53 | 177 | NO: 54 | | | |
| SEQ ID NO: | SEQ ID NO: | SEQ ID | CPR_2 | Q9SB48 | Arabidopsis thaliana |
| 55 | 178 | NO: 56 | | | (Mouse-ear cress) |
| SEQ ID NO: | SEQ ID NO: | SEQ ID | CPR_3 | Q9SUM3 | Arabidopsis thaliana |
| 57 | 179 | NO: 58 | | | (Mouse-ear cress) |
| SEQ ID NO: | SEQ ID NO: | SEQ ID | CPS_SR | O22667 | Stevia rebaudiana |
| 59 | 141 | NO: 60 | | | |
| SEQ ID NO: 61 | SEQ ID NO: 142 | SEQ ID NO: 62 | tCPS_SR | 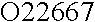 | Stevia rebaudiana |
| SEQ ID NO: | SEQ ID NO: | SEQ ID | KS_SR | Q9XEI0 | Stevia rebaudiana |
| 63 | 143 | NO: 64 | | | |
| SEQ ID NO: 65 | SEQ ID NO: 144 | SEQ ID NO: 66 | tKS_SR | 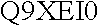 | Stevia rebaudiana |
| SEQ ID NO: | SEQ ID NO: | SEQ ID | KO_SR | Q4VCL5 | Stevia rebaudiana |
| 67 | 145 | NO: 68 | | | |
| SEQ ID NO: 69 | SEQ ID NO: 146 | SEQ ID NO: 70 | KAH_SR | 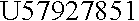 | Stevia rebaudiana |
| SEQ ID NO: | SEQ ID NO: | SEQ ID | UGT1_SR | Q6VAB0 | Stevia rebaudiana |
| 71 | 147 | NO: 72 | | | |
| SEQ ID NO: | SEQ ID NO: | SEQ ID | UGT3_SR | Q6VAA6 | Stevia rebaudiana |
| 73 | 148 | NO: 74 | | | |
| SEQ ID NO: | SEQ ID NO: | SEQ ID | UGT4_SR | Q6VAB4 | Stevia rebaudiana |
| 75 | 149 | NO: 76 | | | |
| SEQ ID NO: | SEQ ID NO: | SEQ ID | CPR_SR | Q216J8 | Stevia rebaudiana |
| 77 | 150 | NO: 78 | | | |
| SEQ ID NO: | | SEQ ID | tHMG1 | G2WJY0 | Saccharomyces |
| 79 | | NO: 80 | | | cerevisiae |
| SEQ ID NO: | | SEQ ID | ERG20 | E7LW73 | Saccharomyces |
| 81 | | NO: 82 | | | cerevisiae |
| SEQ ID NO: | | SEQ ID | BTS1 | E7Q9V5 | Saccharomyces |
| 83 | | NO: 84 | | | cerevisiae |
| SEQ ID NO: | SEQ ID NO: | SEQ ID | KO_Gibfu | O94142 | Gibberella fujikuroi |
| 85 | 180 | NO: 86 | | | |
| SEQ ID NO: 87 | SEQ ID NO: 181 | SEQ ID NO: 88 | UGT2_1a | 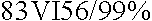 | Stevia rebaudiana |
| SEQ iD NO: | | SEQ ID | KAH_ASR1 | Xxx | S. rebaudiana |
| 89 | | NO: 90 | | | |
| SEQ ID NO: | | SEQ ID | KAH_ASR2 | Q0NZP1_STERE | S. rebaudiana |
| 91 | | NO: 92 | | | |
| SEQ ID NO: | | SEQ ID | KAH_AAT | Q6NKZ8_ARATH | A. thaliana |
| 93 | | NO: 94 | | | |
| SEQ ID NO: 95 | | SEQ ID NO: 96 | KAH_AVV | 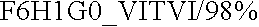 | Vitis vinifera |
| SEQ ID NO: | | SEQ ID | KAH_AMT | Q2MJ20_MEDTR | Medicago truncatula |
| 97 | | NO: 98 | | | |
| SEQ ID NO: 99 | | SEQ ID NO: 100 | UGT2_1b | 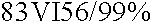 | S. rebaudiana |
| SEQ ID NO: | | SEQ ID | UGT2_2 | Q53UH5_IPOPU | I. purpurea |
| 101 | | NO: 102 | | | |
| SEQ ID NO: 103 | | SEQ ID NO: 104 | UGT2_3 | 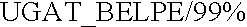 | Bellis perennis |
| SEQ ID NO: | | SEQ ID | UGT2_4 | B3VI56 | S. rebaudiana |
| 105 | | NO: 106 | | | |
| SEQ iD NO: | | SEQ ID | UGT2_5 | Q6VAA8 | S. rebaudiana |
| 107 | | NO: 108 | | | |
| SEQ ID NO: | | SEQ ID | UGT2_6 | Q8LKG3 | S. rebaudiana |
| 109 | | NO: 110 | | | |
| SEQ ID NO: | | SEQ ID | UGT2_7 | B9HSH7_POPTR | Populus trichocarpa |
| 111 | | NO: 112 | | | |
| SEQ ID NO: | | SEQ ID | UGT_RD1 | Q6VAA3 | S. rebaudiana |
| 113 | | NO: 114 | | | |
| SEQ ID NO: | | SEQ ID | UGT_RD2 | Q8H6A4 | S. rebaudiana |
| 115 | | NO: 116 | | | |
| SEQ ID NO: | | SEQ ID | UGT_RD3 | Q6VAA4 | S. rebaudiana |
| 117 | | NO: 118 | | | |
| SEQ ID NO: | | SEQ ID | UGT_RD4 | Q6VAA5 | S. rebaudiana |
| 119 | | NO: 120 | | | |
| SEQ ID NO: | | SEQ ID | UGT_RD5 | Q6VAA7 | S. rebaudiana |
| 121 | | NO: 122 | | | |
| SEQ ID NO: | | SEQ ID | UGT_RD6 | Q6VAA8 | S. rebaudiana |
| 123 | | NO: 124 | | | |
| SEQ ID NO: | | SEQ ID | UGT_RD7 | Q6VAA9 | S. rebaudiana |
| 125 | | NO: 126 | | | |
| SEQ ID NO: | | SEQ ID | UGT_RD8 | Q6VAB1 | S. rebaudiana |
| 127 | | NO: 128 | | | |
| SEQ ID NO: | | SEQ ID | UGT_RD9 | Q6VAB2 | S. rebaudiana |
| 129 | | NO: 130 | | | |
| SEQ ID NO: | | SEQ ID | UGT_RD10 | Q6VAB3 | S. rebaudiana |
| 131 | | NO: 132 | | | |
| SEQ ID NO: | | SEQ ID | UGT_RD11 | B9VVB1 | S. rebaudiana |
| 133 | | NO: 134 | | | |
| SEQ ID NO: | | SEQ ID | UGT_RD12 | C7EA09 | S. rebaudiana |
| 135 | | NO: 136 | | | |
| SEQ ID NO: | | SEQ ID | UGT_RD13 | Q8LKG3 | S. rebaudiana |
| 137 | | NO: 138 | | | |
| SEQ ID NO: | | SEQ ID | UGT_RD14 | B3VI56 | S. rebaudiana |
| 139 | | NO: 140 | | | |
| | SEQ ID NO: | | tCPS | | |
| | 182 | | | | |
| | SEQ ID NO: | | tKS | | |
| | 183 | | | | |
| | SEQ ID NO: | | CPSKS | | |
| | 184 | | | | |
| | SEQ ID NO: | | KAH4 | | |
| | 185 | | | | |
| | SEQ ID NO: | | KO _Gibfu | | |
| | 186 | | | | |
| | SEQ ID NO: | | CPR1 | | |
| | 187 | | | | |
| | SEQ ID NO: | | CPR3 | | |
| | 188 | | | | |
| | SEQ ID NO: | | UGT1 | | |
| | 189 | | | | |
| | SEQ ID NO: | | UGT3 | | |
| | 190 | | | | |
| | SEQ ID NO: | | UGT4 | | |
| | 191 | | | | |
| | SEQ ID NO: | | UGT2 _1a | | |
| | 192 | | | | |
| | SEQ ID NO: | | pTPI | | |
| | 193 | | | | |
| | SEQ ID NO: | | gpdT-pGPD | | |
| | 194 | | | | |
| | SEQ ID NO: | | pgmT-pTEF | | |
| | 195 | | | | |
| | SEQ ID NO: | | pgkT-pPGM | | |
| | 196 | | | | |
| | SEQ ID NO: | | LEU2 and | | |
| | 197 | | flanking | | |
| | | | sequences | | |
| | SEQ ID NO: | | vector | | |
| | 198 | | sequences | | |
| | SEQ ID NO: | | pENO | | |
| | 199 | | | | |
| | SEQ ID NO: | | HPH | | |
| | 200 | | | | |
| SEQ ID NO: | | | Sc Eno2.pro | | |
| 201 | | | | | |
| SEQ ID NO: | | | Sc Fba1.pro | | |
| 202 | | | | | |
| SEQ ID NO: | | | Sc Tef1.pro | | |
| 203 | | | | | |
| SEQ ID NO: | | | Sc Pgk1.pro | | |
| 204 | | | | | |
| SEQ ID NO: | | | KI prom 12.pro | | |
| 205 | | | | | |
| SEQ ID NO: | | | Ag Iox_TEF1.pro | | |
| 206 | | | | | |
| SEQ ID NO: | | | KI prom 6.pro | | |
| 207 | | | | | |
| SEQ ID NO: | | | Sc Pma1.pro | | |
| 208 | | | | | |
| SEQ ID NO: | | | Sc Vps68.pro | | |
| 209 | | | | | |
| SEQ ID NO: | | | Sc Oye2.pro | | |
| 210 | | | | | |
| SEQ ID NO: | | | KANMX ORF | | |
| 211 | | | | | |
| SEQ ID NO: | | | Adh1.ter | | |
| 212 | | | | | |
| SEQ ID NO: | | | Adh2.ter | | |
| 213 | | | | | |
| SEQ ID NO: | | | Gmp1.ter | | |
| 214 | | | | | |
| SEQ ID NO: | | | Sc Tal1.ter | | |
| 215 | | | | | |
| SEQ ID NO: | | | Sc Tpi1.ter | | |
| 216 | | | | | |
| SEQ ID NO: | | | Ag Tef1_Iox.ter | | |
| 217 | | | | | |
| SEQ ID NO: | | | Sc Pdc1.ter | | |
| 218 | | | | | |
| SEQ ID NO: | | | Sc Tdh1.ter | | |
| 219 | | | | | |
| SEQ ID NO: | | | Sc Eno1.ter | | |
| 220 | | | | | |
| SEQ ID NO: | | | KI prom3.pro | | |
| 221 | | | | | |
| SEQ ID NO: | | | KI prom2.pro | | |
| 222 | | | | | |
| SEQ ID NO: | | | Sc PRE3. Pro | | |
| 223 | | | | | |

greyed out ids are truncated and thus a fragment of mentioned UniProt id

1. A product which is a foodstuff, beverage, pharmaceutical composition, tobacco, nutraceutical, oral hygiene composition or cosmetic comprising a sweetener composition, wherein the sweetener composition comprises one or more fermentatively-produced steviol glycoside.

2. A product according to claim 1, wherein the at least one of the one or more fermentatively-produced steviol glycosides is rebaudioside A.

3. A product according to claim 1, wherein the sweetener composition comprises at least about 95% by dry weight of fermentatively-produced rebaudioside A.

4. A product according to claim 1, which is a zero calorie, reduced calorie or diabetic product.

5. A product according to claim 1, which is dedicated for human consumption or is an animal feed or fodder.

6. A product according to claim 1, which is an alcoholic beverages, a natural juices, a carbonated soft drinks, a diet drink, a zero calorie drink, a reduced calorie drink or food, a yogurt drink, an instant juice, an instant coffee, a powdered type of instant beverages, a canned product, a syrup, a fermented soybean paste, a soy sauce, a vinegar, a dressing, a mayonnaise, a ketchup, a curry, a soup, an instant bouillon, a powdered soy sauce, a powdered vinegar, a biscuit, a rice biscuit, a cracker, a bread, a chocolate, a caramel, a candy, a chewing gum, a jelly, a pudding, a preserved fruit or vegetable, a fresh cream, a jam, a marmalade, a flower paste, a powdered milk, an ice cream, a sorbet, a vegetable or fruits packed in a bottle, a canned or boiled bean, a meat or food boiled in sweetened sauce, an agricultural vegetable food product, a seafood, a ham, a sausage, a fish ham, a fish sausage, a fish paste, a deep fried fish product, a dried seafood product, a frozen food product, a preserved seaweed, a preserved meat, a tobacco or a medicinal product.

7. A product according to claim 1, which is a non-carbonated or carbonated beverages optionally comprising a cola, a fruit juices originating in fruits or vegetables, a fruit juices, a fruit juice containing fruit particles, a fruit beverage, a fruit juice beverage, a beverage containing fruit juice, a beverage with fruit flavoring, a vegetable juice, a juice containing vegetables, a mixed juice containing fruit and/or vegetables, a sport drink, an energy drinks, near water or the like drinks, a tea type or favorite type beverage, a beverage containing milk components or a dairy products.

8. A product according to claim 1, wherein the sweetener composition further comprises a natural high intensity sweetener, a synthetic or artificial high intensity sweetener, a natural sweetness suppresser, a umami taste enhancer, an amino acid, a polyamino acid additive, a polyol or sugar alcohol, a reduced calorie sweetener, a carbohydrate, a sugar acid, a flavoring agent, an aroma component, a nucleotide additive, an organic acid additive, an organic acid salt additive, an inorganic acid salt additive, a bitter component additive, an artificial or natural sweetness enhancer, a polymer additive, a protein or protein hydrolyzate additive, a surfactant additive, a flavonoid additive, an alcohol additive, an astringent compound additive, a vitamin, a dietary fiber, an antioxidant, a fatty acid, or a salt.

9. A method for the preparation of a product which is a foodstuff, beverage, pharmaceutical composition, tobacco, nutraceutical, oral hygiene composition or cosmetic comprising a sweetener composition, which method comprises preparing a said product and incorporating a sweetener composition comprising one or more fermentatively-produced steviol glycosides.

10. A method according to claim 9, wherein the product is a product according to claim 1.

11. A sweetener composition comprising one or more fermentatively-produced steviol glycosides used in preparation of a foodstuff, beverage, pharmaceutical composition, tobacco, nutraceutical, oral hygiene composition or cosmetic.

12. Composition comprising a product according to claim 1.

13. Composition according to claim 12 capable of being used to enhance citrus or sour attributes, total aroma impact, sweet aromatic complex, ethyl maltol (strawberry flavor) or brown fruit.

14. A composition which comprises, on a dry solids basis, at least about 60%, at least about 70%, at least about 80%, at least about 90%, at least about 95%, at least about 99% weight of fermentatively-produced Rebaudioside A.
